(19) United States
(12) Patent Application Publication
(10) Pub. No.: US 2025/0064774 A1
(43) Pub. Date: Feb. 27, 2025

(54) FORMULATIONS AND METHODS OF MAKING AND USING THE SAME

(71) Applicant: AraVai Global LLC, Casa Grande, AZ (US)

(72) Inventor: Peter Curtis, Riverside, CA (US)

(21) Appl. No.: 18/725,040

(22) PCT Filed: Dec. 28, 2022
(86) PCT No.: PCT/US2022/054200

§ 371 Date: Jun. 27, 2024

Related U.S. Application Data
(60) Provisional application No. 63/266,250, filed on Dec. 30, 2021.

(51) Int. Cl. A61K 31/352; A61K 31/351; A61K 31/4166; A61K 36/8965; A61P 17/00

(52) U.S. Cl. CPC A61K 31/352; A61K 31/351; A61K 31/4166; A61K 36/8965; A61P 17/00

(57) ABSTRACT

Disclosed herein are skin healing formulations. The skin healing formulations may include a) at least one bioflavonoid, b) at least one saponin, and c) at least one skin soothing agent. Several embodiments also provide use of the skin healing formulations in methods of treating a vascular network of a subject in need thereof.

### INCORPORATION BY REFERENCE TO ANY PRIORITY APPLICATIONS

Any and all applications for which a foreign or domestic priority claim is identified in the PCT Request as filed with the present application are hereby incorporated by reference.

### BACKGROUND

### Field

Disclosed herein are topical formulations (e.g., skin healing formulations) and methods for making and/or using the same. In several embodiments, the topical formulation (e.g., skin healing formulation) includes a) at least one bioflavonoid, b) at least one saponin, and c) at least one skin soothing agent. Also disclosed are methods of ameliorating or treating a vascular network of a subject in need thereof using a skin healing formulation as disclosed herein and methods of making skin healing formulations.

### Description of the Related Art

The lower limbs of the human venous system consist of deep veins and superficial veins. The superficial veins are found in the subcutaneous tissue and drain into the deep veins. Stretching of the superficial veins near the surface of the skin contributes to failure of the venous valves to close properly and allows blood to flow in both directions. The backwards flow of blood is known as venous reflux, and can lead to twisted, bulging veins, a condition known as varicose veins.

Varicose veins affect nearly one third of adults in Western societies and are more frequent in older people as wear and tear on the veins cause their walls to weaken. Many individuals with varicose veins experience discomfort or pain in the vein, sensations such as aching, tightness, burning, itching, or tingling of the legs as well as leg swelling. These symptoms are usually mild in the morning and worsen over the course of a day. In more severe cases, individuals may have skin changes such as an itchy rash or darkening and thinning of the skin of the legs, which can lead to poorly healing sores. Furthermore, in rare cases the veins may burst and bleed quite dramatically.

Surgical treatment options to treat varicose veins include vein stripping, a technique in which insertions are made in the leg, a long wire is inserted into the vein, and the entire vein is removed through the insertions made. However, this procedure may result in scaring and complications such as nerve damage, blood loss, pain, infection, and hematoma can occur, and despite the treatment, a high likelihood remains for developing new varicose veins. Other options to treat varicose veins include sclerotherapy and RF energy and each includes their own drawbacks.

### SUMMARY

In view of the problems with treating varicose veins and the pain associated therewith, a need exists for a non-invasive means for treatment. In several embodiments, provided herein are solutions to one or more problems associated varicose veins as disclosed above or elsewhere herein, their treatment as disclosed above or elsewhere herein, or others. In several embodiments, provided herein are topical formulations (e.g., skin healing formulations), such as roll-on topical compositions, which can improve the condition of varicose veins and/or conditions associated with the presence of varicose veins (e.g., pain, unsightliness, etc.).

Several embodiments disclosed herein pertain to skin healing formulations, their use to treat a vascular network of a subject in need thereof, their methods of manufacture, and their methods of use applied topically to the skin. In several embodiments, the skin healing formulation comprises one or more of a) at least one bioflavonoid, b) at least one saponin, and/or c) at least one skin soothing agent. Several embodiments of skin healing formulations comprise a) at least one bioflavonoid, b) at least one saponin, and c) at least one skin soothing agent, their methods of manufacture, and/or their methods of use in treating a vascular network of a subject in need thereof. In several embodiments, by using one or more of the skin healing formulations described herein applied topically to the skin as a roll-on formulation, a vascular network of a subject can be treated. In several embodiments, the appearance of varicose veins can be improved. In several embodiments, the prominence of varicose veins can be reduced. In several embodiments, pain associated with varicose veins can be alleviated or reduced.

Some embodiments pertain to a skin healing formulation comprising a) at least one bioflavonoid, b) at least one saponin, and c) at least one skin soothing agent. In several embodiments, the skin healing formulation is configured to ameliorate or treat a vascular network of a subject in need thereof.

In several embodiments, the at least one bioflavonoid is configured to provide antioxidant activity. In several embodiments, the at least one bioflavonoid is troxerutin.

In several embodiments, the at least one saponin is configured to provide vasoconstrictor activity, anti-inflammatory activity, keratolytic activity, anti-irritant activity, wound healing activity, and any combination of the foregoing. In several embodiments, the at least one saponin is selected from the group consisting of allantoin, ammonium glycyrrhizate, escin, ruscus aculeatus root, and any combination of the foregoing.

In several embodiments, the at least one skin soothing agent comprises glycoproteins, polysaccharides, and any combination of the foregoing. In several embodiments, the at least one skin soothing agent is acacia senegal gum.

In several embodiments, the formulation further comprises at least one active botanical compound. In several embodiments, the at least one active botanical compound is configured to provide antioxidant activity, moisturizing activity, and any combination of the foregoing. In several embodiments, the at least one active botanical compound is selected from the group consisting of Calendula officinalis, Centella sciatica, and any combination of the foregoing.

In several embodiments, the skin healing formulation further comprises at least one amino acid selected from the group consisting of arginine, aspartic acid, pyroglutamic acid, glycine, alanine, serine, valine, proline, threonine, isoleucine, histidine, phenylalanine, and any combination of the foregoing. In several embodiments, the skin healing formulation further comprises at least one antioxidant selected from the group consisting of tetrahexyldecyl ascorbate, tocopherol and any combination of the foregoing. In several embodiments, the skin healing formulation further comprises at least one emollient selected from the group consisting of butylene glycol, caprylic triglyceride, capric triglyceride, and any combination of the foregoing. In several embodiments, the skin healing formulation further comprises at least one emulsifier wherein the at least one emulsifier is polysorbate 80. In several embodiments, the skin healing formulation further comprises at least one fragrance source wherein the at least one fragrance source is Vibrance Type FW. In several embodiments, the skin healing formulation further comprises at least one humectant selected from the group consisting of glycerine, propylene glycol, propanediol, sodium lactate, sodium pyroglutamic acid, and any combination of the foregoing. In several embodiments, the skin healing formulation further comprises at least one moisturizer selected from the group consisting of panthenol, hydrolyzed yeast protein, cholecalciferol, and any combination of the foregoing. In several embodiments, the skin healing formulation further comprises at least one rubifacient wherein the at least one rubifacient is methyl nicotinate. In several embodiments, the skin healing formulation further comprises at least one antimicrobial selected from the group consisting of phenyl propanol, caprylyl glycol and any combination of the foregoing.

In several embodiments, a subgroup of the components includes troxerutin, escin, ruscus aculeatus root, and acacia senegal gum. In several embodiments, the sum of troxerutin, escin, ruscus aculeatus root, and acacia senegal gum, (i.e., the sum of components of the subgroup), adds up to 100 wt %. In several embodiments, troxerutin is present at a weight percent from about 10% to about 98 wt % based on a total weight of a subgroup. In several embodiments, escin is present at a weight percent from about 0.5% to about 40 wt % based on a total weight of a subgroup. In several embodiments, ruscus aculeatus root is present at a weight percent from about 0.3% to about 20 wt % based on a total weight of a subgroup. In several embodiments, acacia senegal gum is present at a weight percent from about 2% to about 40 wt % based on a total weight of a subgroup.

In several embodiments, troxerutin is present at a weight percent from about 0.01% to about 30% based on a total weight of the skin healing formulation. In several embodiments, escin is present at a weight percent from about 0.005% to about 5% based on a total weight of the skin healing formulation. In several embodiments, ruscus aculeatus root is present at a weight percent from about 0.001% to about 3% based on a total weight of the skin healing formulation. In several embodiments, acacia senegal gum is present at a weight percent from about 0.02% to about 4% based on a total weight of the skin healing formulation.

In several embodiments, based on a total weight of the skin healing formulation, troxerutin is present at a weight percent from about 0.95% to about 1.05%; Ginkgo biloba is present at a weight percent from about 0.19% to about 0.21%; escin is present at a weight percent from about 0.14% to about 0.16%; ruscus aculeatus root is present at a weight percent from about 0.074% to about 0.076%; allantoin is present at a weight percent from about 0.38% to about 0.42%; ammonium glycyrrhizate is present at a weight percent from about 0.048% to about 0.052%; acacia senegal gum is present at a weight percent from about 0.24% to about 0.26%; xanthan gum is present at a weight percent from about 0.20% to about 0.22%; Calendula officinalis is present at a weight percent from about 0.017% to about 0.018%; centella asiatica is present at a weight percent from about 0.032% to about 0.033%; arginine is present at a weight percent from about 0.038% to about 0.042%; aspartic acid is present at a weight percent from about 0.025% to about 0.027%; pyroglutamic acid is present at a weight percent from about 0.020% to about 0.022%; glycine is present at a weight percent from about 0.0061% to about 0.0067%; alanine is present at a weight percent from about 0.0058% to about 0.0062%; serine is present at a weight percent from about 0.0038% to about 0.0042%; valine is present at a weight percent from about 0.0031% to about 0.0033%; proline is present at a weight percent from about 0.0019% to about 0.0021%; threonine is present at a weight percent from about 0.0019% to about 0.0021%; isoleucine is present at a weight percent from about 0.0019% to about 0.0021%; histidine is present at a weight percent from about 0.00076% to about 0.00084%; phenylalanine is present at a weight percent from about 0.00076% to about 0.00084%; tetrahexyldecyl ascorbate is present at a weight percent from about 0.010% to about 0,012%; tocopherol is present at a weight percent from about 0.001% to about 0.0012%; butylene glycol is present at a weight percent from about 3.3% to about 3.7%; caprylic triglyceride is present at a weight percent from about 2.4% to about 2.6%; capric triglyceride is present at a weight percent from about 2.4% to about 2.6%; polysorbate 80 is present at a weight percent from about 0.95% to about 1.05%; Vibrance Type FW is present at a weight percent from about 0.16% to about 0.18%; glycerin is present at a weight percent from about 6.1% to about 6.7%; propylene glycol is present at a weight percent from about 2.4% to about 2.6%; propanediol is present at a weight percent from about 0.26% to about 0.28%; sodium lactate is present at a weight percent from about 0.057% to about 0.063%; sodium pyroglutamic acid is present at a weight percent from about 0.071% to about 0.079%; panthenol is present at a weight percent from about 0.48% to about 0.52%; hydrolyzed yeast protein is present at a weight percent from about 0.019% to about 0.021%; cholecalciferol is present at a weight percent from about 0.0000056% to about 0.000006%; methyl nicotinate is present at a weight percent from about 1.05% to about 1.15%; phenyl propanol is present at a weight percent from about 0.5% to about 0.6%; caprylyl glycol is present at a weight percent from about 0.26% to about 0.3%; and water is present at a weight percent from about 71.8% to about 86.8%.

In several embodiments, the skin healing formulation is applied topically to the skin of a subject in need thereof.

In several embodiments, the skin healing formulation is configured to be applied as a roll-on formulation.

Some embodiments pertain to a method of ameliorating or treating a vascular network of a subject in need thereof. In several embodiments, the method further comprises improving the appearance of varicose veins and/or reducing the prominence of varicose veins of the subject. In several embodiments, the method further comprises alleviating or reducing pain of varicose veins of the subject.

Some embodiments pertain to a method of ameliorating or treating pain of a subject in need thereof. In several embodiments, the method further comprises reducing or lessening the pain of the subject. In several embodiments, the method further comprises reducing or lessening the pain caused by nerve cells and/or neurons (e.g., sciatic nerve), of the subject. In several embodiments, the method further comprises treating, the extremities of the subject to reduce or lessen the pain of the subject. In several embodiments, the method further comprises treating the extremities of the subject to reduce or lessen the pain caused by nerve cells and/or neurons (e.g., sciatic nerve), of the subject.

Surprisingly, it has been found that pain may be treated at an area located a distance away from the site of application of the formulation. For example, application of the formulation to the crotch of the knee or lower leg can result in alleviation of pain at the site of application as well as other areas (e.g., where the formulation was not directly applied). Taking the trunk of the body to be a spatial reference point along with the site of application, in several embodiments, application to a site on an appendage may treat and/or alleviate pain that is proximally located relative to the site of treatment. For example, the pain or discomfort may be alleviated at a site along the appendage between the site of application and the trunk of the body (and/or on the trunk of the body). For example, application of the formulation at the upper leg or behind the knee may result in lessened pain at the lower back. Additionally or alternatively, in several embodiments, application to a site on an appendage may treat and/or alleviate pain that is distally located relative to the site of treatment (e.g., between the site of application and an extremity or terminus of the appendage). For example, application of the formulation at the upper leg or behind the knee may result in lessened pain at the site of treatment as well as in the lower leg and foot.

In several embodiments, the method comprises administering to the subject a skin healing formulation comprising, a) at least one bioflavonoid, b) at least one saponin, and c) at least one skin soothing agent. In several embodiments, the at least one bioflavonoid is troxerutin. In several embodiments, the at least one saponin is selected from the group consisting of allantoin, ammonium glycyrrhizate, escin, ruscus aculeatus root, and any combination of the foregoing. In several embodiments, the at least one skin soothing agent is acacia senegal gum.

In several embodiments of the method, the formulation further comprises at least one active botanical compound. In several embodiments, the at least one active botanical compound is selected from the group consisting of Calendula officinalis, Centella sciatica, and any combination of the foregoing.

Some embodiments pertain to a skin healing formulation that may include one or more active ingredients. In several embodiments, as disclosed elsewhere herein, an active ingredient may include a compound configured to provide antioxidant activity (e.g., an antioxidant), skin soothing activity (e.g., a skin soothing agent), and/or moisturizing activity (e.g., a moisturizer). In several embodiments, the formulation comprises at least a first active ingredient. In several embodiments, the formulation further comprises a second active ingredient. In several embodiments, the formulation further comprises additional active ingredients (a third, fourth, fifth, sixth, etc.). In several embodiments, the active ingredient (or active ingredients) is selected from the group consisting of an active botanical compound, an amino acid, an antimicrobial, an antioxidant, a bioflavonoid, an emollient, a humectant, a moisturizer, a rubifacient, a saponin, a skin soothing agent, and/or combinations of any of the foregoing. In several embodiments, the active ingredient (or active ingredients) is selected from the group consisting of Calendula officinalis flower extract, centella asiatica extract, arginine, aspartic acid, pyroglutamic acid, glycine, alanine, serine, valine, proline, threonine, isoleucine, histidine, phenylalanine, tetrahexyldecyl ascorbate, phenyl propanol, caprylyl glycol, tocopherol, troxerutin, Ginkgo biloba leaf extract, capric triglyceride, caprylic triglyceride, butylene glycol, glycerin, propylene glycol, propanediol, sodium pyroglutamic acid, sodium lactate, panthenol, hydrolyzed yeast protein, cholecalciferol, methyl nicotinate, escin, ruscus aculeatus root extract, allantoin, ammonium glycyrrhizate, acacia senegal gum, xanthan gum, and/or combinations of any of the foregoing.

In several embodiments, the formulation comprises at least a first inactive ingredient. In several embodiment, the formulation further comprises a second inactive ingredient. In several embodiments, the formulation further comprises additional inactive ingredients (a third, fourth, fifth, sixth, etc.). In several embodiments, the inactive ingredient (or inactive ingredients) is selected from the group consisting of an emulsifier, a fragrance, and/or combinations of any of the foregoing. In several embodiments, the inactive ingredient (or inactive ingredients) is selected from the group consisting of polysorbate 80, fragrance, lactic acid, water, and/or combinations of any of the foregoing.

In several embodiments of the method, the skin healing formulation further comprises at least one amino acid selected from the group consisting of arginine, aspartic acid, pyroglutamic acid, glycine, alanine, serine, valine, proline, threonine, isoleucine, histidine, phenylalanine, and any combination of the foregoing. In several embodiments, the skin healing formulation further comprises at least one antioxidant selected from the group consisting of tetrahexyldecyl ascorbate, tocopherol and any combination of the foregoing. In several embodiments, the skin healing formulation further comprises at least one emollient selected from the group consisting of butylene glycol, caprylic triglyceride, capric triglyceride, and any combination of the foregoing. In several embodiments, the skin healing formulation further comprises at least one emulsifier wherein the at least one emulsifier is polysorbate 80. In several embodiments, the skin healing formulation further comprises at least one fragrance source wherein the at least one fragrance source is Vibrance Type FW. In several embodiments, the skin healing formulation further comprises at least one humectant selected from the group consisting of glycerine, propylene glycol, propanediol, sodium lactate, sodium pyroglutamic acid, and any combination of the foregoing. In several embodiments, the skin healing formulation further comprises at least one moisturizer selected from the group consisting of panthenol, hydrolyzed yeast protein, cholecalciferol, and any combination of the foregoing. In several embodiments, the skin healing formulation further comprises at least one rubifacient wherein the at least one rubifacient is methyl nicotinate. In several embodiments, the skin healing formulation further comprises at least one antimicrobial selected from the group consisting of phenyl propanol, caprylyl glycol and any combination of the foregoing.

In several embodiments, the formulation is administered topically. In several embodiments, the formulation comprises a roll-on formulation.

In several embodiments, a delay time after the formulation is administered that the improved appearance begins is equal to or less than about 10 seconds to about 10 minutes. In several embodiments, a time span after the formulation is administered that the improved appearance ends is equal to or more than about 30 minutes to about 8 hours.

In several embodiments, the subject is a mammal. In several embodiments, the subject is a human.

### BRIEF DESCRIPTION OF THE DRAWINGS

FIG. 1A depicts a posterior view of the lower leg of a subject before treatment.

FIG. 1B depicts the lower leg of the subject 20 minutes after a first treatment with an embodiment of the skin healing formulation.

FIG. 1C depicts the lower leg of the subject one hour after the first treatment with an embodiment of the skin healing formulation and immediately after a second treatment with an embodiment of the skin healing formulation.

FIG. 1D depicts the lower leg of the subject eight hours after the second treatment with an embodiment of the skin healing formulation.

FIG. 2A depicts the lower leg of the subject one month after the second treatment with an embodiment of the skin healing formulation and 25 seconds after a third treatment with an embodiment of the skin healing formulation.

FIG. 2B depicts the lower leg of the subject 20 minutes after a third treatment with an embodiment of the skin healing formulation.

FIG. 3A depicts the lower leg of the subject receiving a fourth treatment with an embodiment of the skin healing formulation at a time 22 minutes after the third treatment with an embodiment of the skin healing formulation.

FIG. 3B depicts the lower leg of the subject 18 minutes after the fourth treatment with an embodiment of the skin healing formulation.

FIG. 3C depicts the lower leg of the subject 26 minutes after the fourth treatment with an embodiment of the skin healing formulation.

### DETAILED DESCRIPTION

Several embodiments disclosed herein provide skin healing formulations. Several embodiments also provide use of the skin healing formulations in methods of treating a vascular network of a subject in need thereof. In several embodiments, the formulations (and their methods of use) improve the appearance of varicose veins and/or reducing the prominence of varicose veins of the subject. In several embodiments, the formulations (and their methods of use) are configured to alleviate and/or do alleviate and/or reduce pain associated with varicose veins of the subject. In several embodiments, the skin healing formulation, includes at least one bioflavonoid, at least one saponin, and at least one skin soothing agent. The following description provides context and examples, but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

As used herein, the term “acacia senegal gum” is given its plain and ordinary meaning. A “acacia senegal gum” may include a dried exudate (i.e., hardened sap) of two species of the acacia (sensu lato) tree: Acacia senegal, that is also known as Senegalia senegal, and Vachellia (Acacia) seyal. The sap is also known as Gum arabic, gum sudani, acacia gum, Arabic gum, gum acacia, acacia, Senegal gum, Indian gum, and includes a complex mixture of glycoproteins and polysaccharides predominantly consisting of arabinose and galactose.

As used herein, the term “anti-inflammatory” is the property of a substance or treatment that reduces inflammation or swelling.

As used herein, the term “antioxidant” a substance or treatment that reduces or inhibits chemical reactions that can produce free radicals and chain reactions that may damage cells of the body.

As used herein, the term “bioflavonoid” is a member of the class of polyphenolic secondary metabolites found in plants, and thus commonly consumed in the diets of humans.

As used herein, the term “emollient” is a substance that protects, moisturizes, and lubricates the skin.

As used herein, the term “emulsifier” is a substance that stabilizes an emulsion by increasing its kinetic stability. Emulsifiers are typically amphiphilic.

As used herein, the term “escin” (or “aescin”) is a substance derived from Aesculus hippocastanum, the horse chestnut which is a species of flowering plant in the soapberry and lychee family Sapindaceae. Escin may include one or more of α-escin, β-escin, α-aescin, and β-aescin.

As used herein, the term “glycoprotein” is a proteins which contain oligosaccharide chains (glycans) covalently attached to amino acid side-chains. The carbohydrate is attached to the protein in a cotranslational or posttranslational modification.

As used herein, the term “humectant” is a hygroscopic substance used to attract and retain the moisture in the air nearby via absorption. Humectants can be used in topical dosage forms to increase the solubility of a chemical compound's active ingredients, increasing the active ingredients' ability to penetrate skin, or its activity time.

As used herein, the term “keratolytic” is a substance used to soften keratin, a major component of the skin. Keratolytic agents can be applied to a lesion on the skin in order to thin the skin on and around it. This therapy causes the outer layer of the skin to loosen and shed.

As used herein, the term “rubifacient” is a substance for external application that produces redness of the skin.

As used herein, the term “ruscus aculeatus root” is one of a group of saponins that includes ruscogenins, ruscogenen and neoruscogenin, that are found in the root of Ruscus aculeatus (butcher's broom).

As used herein, the term “polysaccharide” is one of a group of carbohydrates (e.g. starch, cellulose, or glycogen) whose molecules consist of a number of sugar molecules bonded together.

As used herein, a “saponin” is given its plain and ordinary meaning. A saponin is a triterpene glycoside. They are widely distributed but found particularly in soapwort, a flowering plant, and the soapbark tree.

As used herein, the term “troxerutin” is a bioflavonoid isolated from Sophora japonica that is also known as hydroxyethylrutoside. Troxerutin has antioxidant and vasoprotective properties.

As used herein, the term “vasoconstrictor” is a substance that causes a narrowing of the blood vessels resulting from contraction of the muscular wall of the vessels.

As used herein, “subject,” “host,” “patient,” and “individual” are used interchangeably and shall be given its ordinary meaning and shall also refer to an organism that is treated. This includes mammals, e.g., a human, a non-human primate, ungulates, canines, felines, equines, mice, rats, and the like. The term “mammal” includes both human and non-human mammals.

“Diagnosis” as used herein shall be given its ordinary meaning and shall also include determination of a subject's susceptibility to a disease or disorder, determination as to whether a subject is presently affected by a disease or disorder, prognosis of a subject affected by a disease or disorder (e.g., identification of cancer or cancerous states, stages of cancer, or responsiveness of cancer to therapy), and use of therametrics (e.g., monitoring a subject's condition to provide information as to the effect or efficacy of therapy).

The term “sample” or “biological sample” shall be given its ordinary meaning and also encompasses a variety of sample types obtained from an organism and can be used in an imaging, a diagnostic, a prognostic, or a monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom. Symptoms of varicose veins may include, for example, visible, blue, and/or enlarged veins in your legs; aching pain (especially after long periods of standing or sitting); throbbing or cramping in the thigh or calf; a feeling of heaviness in the leg(s); swelling in the lower leg, ankle, or foot; itching in the affected limb; or others.

The term “therapeutically effective amount,” as used herein, refers to an amount of the therapeutic (e.g., skin healing formulation) that imparts a modulating effect, which, for example, can be a beneficial effect, to a subject afflicted with a disorder, disease or illness, including improvement in the condition of the subject (e.g., modulating one or more symptoms), delay or reduction in the progression of the condition, prevention or delay of the onset of the disorder, and/or change in clinical parameters, disease or illness, etc. For example, in some embodiments, an effective amount can refer to the amount of skin healing formulation that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or ranges spanning and/or including the aforementioned values. Actual dosage levels of active ingredients and agents in an active skin healing formulation of the disclosed subject matter can be varied so as to administer an amount of the active agent(s) that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. Determination and adjustment of an effective dose, as well as evaluation of when and how to make such adjustments, are contemplated herein. The term “a therapeutically effective amount” can mean an amount of skin healing formulation sufficient to prevent the spread of or reverse varicose vein development.

The term “control” refers shall be given its ordinary meaning and shall also include a sample or standard used for comparison with a sample which is being examined, processed, characterized, analyzed, etc. In several embodiments, the control is a sample obtained from a healthy patient or a non-tumor tissue sample obtained from a patient diagnosed with a tumor. In several embodiments, the control is a historical control or standard reference value or range of values. In several embodiments, the control is a comparison to a wild-type arrangement or scenario.

As used herein, the term “weight percent,” when referring to a component, is the weight of the component divided by the weight of the composition that includes the component, multiplied by 100%. For example the weight percent of component A when 5 grams of component A is added to 95 grams of component B is 5% (e.g., 5 g A/(5 g A+95 gB)×100%). Where a % is provided without specifying how the percent was calculated (e.g., by weight, by volume, etc.), weight percent is what is meant.

As used herein, when the term “collectively or individually” (and variations thereof) modifies an amount of a component or components (e.g., a weight percent) of multiple component composition, this usage means that each individual component may be provided in the amount disclosed or that combined amount of components may be provided in the amount disclosed. For example, if agents A and B are referred to as, collectively or individually, being present in a composition at a wt % 5%, that means that A may be at 5 wt % in the composition (individually), B may be at 5 wt % in the composition (individually), or the combination of A and B may be present at a total of 5 wt % (A+B=5 wt %, e.g., collectively). Where A is present at 5 wt %, B may be absent. Where B is present at 5 wt %, A may be absent. Alternatively, where both A and B are present, A may be at 5 wt % (individually) and B may be at 5 wt % (individually), totaling 10 wt % (collectively).

When referring to various features, the terms “or ranges including and/or spanning the aforementioned values” may be used. These terms (and variations thereof) are meant to include any range that includes or spans any of the aforementioned values. For example, with regard to the concentration for an ingredient, the wt % of that ingredient may be expressed as “equal to or at least about: 1%, 5%, 10%, 20%, or ranges including and/or spanning the aforementioned values.” This language includes not only the particular wt % provided and the range exceeding that value (e.g., equal to or at least about 1%, equal to or at least about 5%, equal to or at least about 10%, and equal to or at least about 20%) but also the wt % ranges for the ingredient spanning those values (e.g., from 1% to 20%, 1% to 10%, 1% to 5%, 5% to 20%, 5% to 10%, and 10% to 20%). Similarly, with regard to the concentration for an ingredient, the wt % that ingredient may be expressed as “equal to or less than about: 1%, 5%, 10%, 20%, or ranges including and/or spanning the aforementioned values.” This language includes not only the particular wt % provided and the range below that value (e.g., equal to or less than about 1%, equal to or less than about 5%, equal to or less than about 10%, and equal to or less than about 20%) but also the wt % ranges for the ingredient spanning those values (e.g., from 1% to 20%, 1% to 10%, 1% to 5%, 5% to 20%, 5% to 10%, and 10% to 20%).

As used herein, an “amino acid” includes amino acids with natural amino acid side chains or non-natural amino acid side chains. As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine. As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives. Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising” “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### INTRODUCTION

Individuals with varicose veins experience discomfort or pain in the vein, sensations such as aching, tightness, burning, itching, or tingling of the legs as well as leg swelling. Surgical and other treatment options for varicose veins are limited and few, if any, non-invasive treatments of varicose veins exist. There remains a need for a topical skin healing formulation that can be used in a method of ameliorating or treating a vascular network of a subject in need thereof and there remains a need for a topical skin healing formulation to be used for the treatment of a vascular network which offers one or more advantages over current formulations. Those advantages include: improving the appearance of varicose veins of the subject; reducing the prominence of varicose veins of the subject; and alleviating or reducing pain of varicose veins of the subject. Several embodiments disclosed herein pertain to skin healing formulations that achieve one or more of these advantages (or others).

### Skin Healing Formulation

As disclosed elsewhere herein, in several embodiments, the skin healing formulation may include one or more active ingredients. In several embodiments, as disclosed elsewhere herein, an active ingredient may include a compound configured to provide antioxidant activity (e.g., an antioxidant), skin soothing activity (e.g., a skin soothing agent), and/or moisturizing activity (e.g., a moisturizer). In several embodiments, the formulation comprises at least a first active ingredient. In several embodiments, the formulation further comprises a second active ingredient. In several embodiments, the formulation further comprises additional active ingredients (a third, fourth, fifth, sixth, etc.).

In several embodiments, the skin healing formulation is configured to ameliorate or treat a vascular network of a subject in need thereof. As used herein, the term “vascular network” includes veins, arteries, capillaries, varicose veins, spider veins, or combinations of any of the foregoing. As used herein, the term “varicose veins” are large, raised, swollen blood vessels that can twist and turn. Varicose veins can be present in the legs and can be seen through the skin. As used herein, the term “spider veins” are smaller, red, purple, and blue vessels that also can twist and turn. Spider veins can be present in the legs chest, and face and can be seen through the skin.

As disclosed elsewhere herein, in several embodiments, the skin healing formulation may include one or more bioflavonoids. In several embodiments, the bioflavonoid is configured to provide antioxidant activity. In several embodiments, the bioflavonoid comprises, consists of, or consists essentially of a Ginkgo biloba and/or troxerutin. In several embodiments, the bioflavonoid is selected from the group consisting of troxerutin, Ginkgo biloba, or combinations of any of the foregoing. In several embodiments, the bioflavonoid (or bioflavonoids), collectively or individually, are present in the skin healing formulation at a weight percent of equal to or less than about: 0.01%, 0.05%, 0.1%, 0.2%, 0.21%, 0.22%, 0.23%, 0.3% 05%, 08%, 0.9%, 1.0%, 1.1%, 1.2%, 1.3%, 2%, 5%, 7.5%, 10%, 15%, 20%, 25%, 30%, or ranges including and/or spanning the aforementioned values.

In several embodiments, as disclosed elsewhere herein, the skin healing formulation comprises troxerutin. In several embodiments, the troxerutin is present in the skin healing formulation at a weight percent of equal to or less than about: 0.01%, 0.5%, 0.8%, 0.9%, 0.95%, 0.98%, 1.0%, 1.02%, 1.05%, 1.1%, 1.15%, 1.16%, 1.17%. 1.18%, 1.19%, 1.2%, 1.21%, 1.23%, 1.25%, 1.3%, 2%, 5%, 7.5%, 10%, 15%, 20%, 25%, 30%, or ranges including and/or spanning the aforementioned values.

In several embodiments, as disclosed elsewhere herein, the skin healing formulation comprises Ginkgo biloba. In several embodiments, the Ginkgo biloba is present in the skin healing formulation at a weight percent of equal to or less than about: 0.01%, 0.05%, 0.1%, 0.15%, 0.17%, 0.18%, 0.19%, 0.2%, 0.21%, 0.22%, 0.23%, 0.3%, 0.5%, 1%, 2%, 5%, 7.5%, 1%, 2%, 5%, 7%, 10%, or ranges including and/or spanning the aforementioned values.

As disclosed elsewhere herein, in several embodiments, the skin healing formulation may include one or more saponin source and/or one or more saponin. In several embodiments, a saponin source (or a saponin) may be configured to provide vasoconstrictor activity, anti-inflammatory activity, keratolytic activity, anti-irritant activity, wound healing activity, or any combination of the foregoing. In several embodiments, the saponin source is selected from the group consisting of escin, ruscus aculeatus root, allantoin, ammonium glycyrrhizate, and any combination of the foregoing. In several embodiments, the saponin source (or saponin sources), collectively or individually, are present in the skin healing formulation at a weight percent of equal to or less than about: 0.01%, 0.05%, 0.1%, 0.2%, 0.3%, 0.4%, 0.5%, 055%, 0.58%, 0.6%, 0.62%, 0.64%, 0.66%, 0.67%, 0.673%, 0.676%, 0.68%, 0.683%, 0.686%, 0.69%, 0.7%, 0.75%, 0.8% 0.9%, 1.0%, 1.5%, 3%, 5%, 10%, or ranges including and/or spanning the aforementioned values.

In several embodiments, as disclosed elsewhere herein, the skin healing formulation comprises escin. In several embodiments, escin is present in the skin healing formulation at a weight percent of equal to or less than about: 0.005%, 0.01%, 0.05%, 0.08%, 0.09%, 0.1%, 0.11%, 0.12%, 0.13%, 0.14%, 0.143%, 0.145% 0.147%, 0.15%, 0.152%, 0.155%, 0.158%, 0.16%, 0.17%, 0.18%, 0.2%, 0.5%, 1%, 5%, ranges including and/or spanning the aforementioned values.

In several embodiments, as disclosed elsewhere herein, the skin healing formulation comprises ruscus aculeatus root. In several embodiments, the ruscus aculeatus root is present in the skin healing formulation at a weight percent of equal to or less than about: 0.001%, 0.005%, 0.01%, 0.03%, 0.04%, 0.05%, 0.06%, 0.07%, 0.072%, 0.073%, 0.074%, 0.0745%, 0.0748%, 0.075%, 0.0752%, 0.0755%, 0.0758%, 0.076%, 0.077%, 0.079%, 0.08%, 0.09%, 0.1%, 0.15%, 0.2%, 0.5%, 1%, 1.5%, 2%, 2.5%, 3%, or ranges including and/or spanning the aforementioned values.

In several embodiments as disclosed elsewhere herein, the skin healing formulation comprises allantoin. In several embodiments, the allantoin is present in the skin healing formulation at a weight percent of equal to or less than about: 0.01%, 0.05%, 0.1%, 0.2%, 0.3%, 0.35%, 0.38%, 0.39%, 0.4%, 0.41%, 0.42%, 0.43%, 0.45%, 0.5%, 0.55%, 0.6%, 0.65%, 00.7%, 0.8%, 0.9%, 1.0%, 1.5%, 3%, 5%, or ranges including and/or spanning the aforementioned values.

In several embodiments, as disclosed elsewhere herein, the skin healing formulation comprises ammonium glycyrrhizate. In several embodiments, the ammonium glycyrrhizate is present in the skin healing formulation at a weight percent of equal to or less than about: 0.001%, 0.005%, 0.01%, 0.03%, 0.04%, 0.045%, 0.046%, 0.047%, 0.048%, 0.049%, 0.05%, 0.051%, 0.052%, 0.053%, 0.054%, 0.055%, 0.06%, 0.07%, 0.08%, 0.09%, 0.1%, 0.15%, 0.2%, 0.5%, 1%, 5%, or ranges including and/or spanning the aforementioned values.

As disclosed elsewhere herein, in several embodiments, the skin healing formulation may include one or more skin soothing agent. In several embodiments, the skin soothing agent comprises glycoproteins, polysaccharides, and any combination of the foregoing. In several embodiments, the skin soothing agent is selected from the group consisting of acacia senegal gum, xanthan gum, and any combination of the foregoing. In several embodiments, the skin soothing agent (or skin soothing agents), collectively or individually, are present in the skin healing formulation at a weight percent of equal to or less than about: 0.01%, 0.05%, 0.1%, 0.2%, 0.3%, 0.35%, 0.38%, 0.39%, 0.4%, 0.41%, 0.42%, 0.43%, 0.44%, 0.443%, 0.446%, 0.45%, 0.453%, 0.456%, 0.46%, 0.47%, 0.48%, 049%, 0.5%, 0.55%, 0.6%, 0.65%, 0.7%, 0.8%, 0.9%, 1.0%, 1.5%, 3% 5%, or ranges including and/or spanning the aforementioned values.

In several embodiments, the skin healing formulation comprises acacia senegal gum. In several embodiments, the acacia senegal gum is present in the skin healing formulation at a weight percent of equal to or less than about: 0.01%, 0.05%, 0.08%, 0.09%, 0.1%, 0.15%, 0.2%, 0.21%, 0.22%, 0.23%, 0.24%, 0.243%, 0.246%, 0.25%, 0.253%, 0.256%, 0.26%, 0.27%, 0.28%, 0.29%, 0.3%, 0.5%, 1%, 2%, 2.5%, 3%, 3.5%, 4%, or ranges including and/or spanning the aforementioned values.

In several embodiments, the skin healing formulation comprises xanthan gum. In several embodiments, the xanthan gum is present in the skin healing formulation at a weight percent of equal to or less than about: 0.001%, 0.005%, 0.01%, 0.03%, 0.04%, 0.05%, 0.1%, 0.15%, 0.16%, 0.17%, 0.18%, 0.19%, 0.193%, 0.196%, 0.2%, 0.203%, 0.206%, 0.21%, 0.22%, 0.23%, 0.24%, 0.25%, 0.3%, 0.5%, 1%, 5%, or ranges including and/or spanning the aforementioned values.

As disclosed elsewhere herein, in several embodiments, the skin healing formulation may include one or more active botanical compound. In several embodiments, the active botanical compound is configured to provide antioxidant activity, moisturizing activity, and any combination of the foregoing. In several embodiments, the active botanical compound is selected from the group consisting of Calendula officinalis, Centella sciatica, and any combination of the foregoing (though Ginkgo biloba is, in actuality, a botanical, for the purpose of this disclosure and ingredient ratios disclosed herein, it is classified as a bioflavonoid). In several embodiments, the active botanical compound (or active botanical compounds), collectively or individually, are present in the skin healing formulation at a weight percent of equal to or less than about: 0.01%, 0.02%, 0.04%, 0.043%, 0.046%, 0.05%, 0.053%, 0.056%, 0.06%, 0.08%, 0.09%, 0.1%, 0.15%, 0.2%, 021%, 0.22%, 0.23%, 24%, 0.25%, 0.26%, 0.27%, 0.28%, 0.29%, 0.3%, 0.5%, 1%, 5%, or ranges including and/or spanning the aforementioned values.

In several embodiments, as disclosed elsewhere herein, the skin healing formulation comprises Calendula officinalis. In several embodiments, the Calendula officinalis is present in the skin healing formulation at a weight percent of equal to or less than about: 0.0005%, 0.001% z, 0.003%, 0.004%, 0.005%, 0.01%, 0.015%, 0.016%, 0.017%, 0.0172%, 0.0174%, 0.0176%, 0.0178%, 0.018%, 0.019%, 0.02%, 0.021%, 0.022%, 0.023%, 0.025%, 0.05%, 0.1%, 0.5%, 1%, 5%, or ranges including and/or spanning the aforementioned values.

In several embodiments, as disclosed elsewhere herein, the skin healing formulation comprises (Centella sciatica. In several embodiments, the Centella sciatica is present in the skin healing formulation at a weight percent of equal to or less than about: 0.001%, 0.005%, 0.01%, 0.02%, 0.025%, 0.028%, 0.029%, 0.0295%, 0.03%, 0.0305%, 0.031%, 0.0315%, 0.032%, 0.0323%, 0.0326%, 0.033%, 0.0333%, 0.0336%, 0.034%, 0.035%, 0.04%, 0.05%, 0.1% 0.5%, 1%, 5%, 10%, 15%, 20%, or ranges including and/or spanning the aforementioned values.

As disclosed elsewhere herein, in several embodiments, the skin healing formulation may include one or more amino acids. In several embodiments, the amino acid is present as a salt form. In several embodiments, the amino acid is selected from the group consisting of arginine, aspartic acid, pyroglutamic acid, glycine, alanine, serine, valine, proline, threonine, isoleucine, histidine, phenylalanine, and any combination of the foregoing. In several embodiments, the amino acid (or amino acids), collectively or individually, are present in the skin healing formulation at a weight percent of equal to or less than about: 0.01%, 0.05%, 0.08%, 0.09%, 0.1%, 0.11%, 0.12%, 0.12%, 0.13%, 0.14%, 0.15%, 0.2%, 0.25%, 0.3%, 0.5%, 1%, 5%, or ranges including and/or spanning the aforementioned values.

In several embodiments, as disclosed elsewhere herein, the skin healing formulation comprises glycine. In several embodiments, the glycine is present in the skin healing formulation at a weight percent of equal to or less than about: 0.0001%, 0,0005%, 0,001%, 0.003%, 0.004%, 0.005%, 0.0055%, 0.0058%, 0.006%, 00062%, 0.0064%, 0.0066%, 0.0068%, 0.007%, 0.01%, 0.015%, 0.02%, 0.05%, 0.1%, 0.5%, 1%, 5%, or ranges including and/or spanning the aforementioned values.

As disclosed elsewhere herein, in several embodiments, the skin healing formulation may include one or more antioxidant. In several embodiments, the antioxidant is selected from the group consisting of tetrahexyldecyl ascorbate, tocopherol, and any combination of the foregoing. (though Ginkgo biloba has antioxidant properties, for the purpose of this disclosure and ingredient ratios, it is classified as a bioflavonoid and not an antioxidant). In several embodiments, the antioxidant (or antioxidants), collectively or individually, are present in the skin healing formulation at a weight percent of equal to or less than about: 0.001%, 0.005%, 0.008%, 0.009%, 0.01%, 0.011%, 0.012%, 0.013%, 0.014%, 0.015%, 0.02%, 0.03%, 0.05%, 0.1%, 0.5%, 1%, 1.5%, 2%, or ranges including and/or spanning the aforementioned values.

In several embodiments, as disclosed elsewhere herein, the skin healing formulation comprises tetrahexyldecyl ascorbate. In several embodiments, the tetrahexyldecyl ascorbate is present in the skin healing formulation at a weight percent of equal to or less than about: 0.001%, 0.005%, 0.008%, 0.009%, 0.01%, 0.011%, 0.012%, 0.013%, 0.014%, 0.015%, 0.02%, 0.03%, 0.05%, 0.1%, 0.5%, 1%, 1.5%, 2%, or ranges including and/or spanning the aforementioned values.

In several embodiments, as disclosed elsewhere herein, the skin healing formulation comprises tocopherol. In several embodiments, the tocopherol is present in the skin healing formulation at a weight percent of equal to or less than about: 0.0001%, 0.0005%, 0.0008%, 0.0009%, 0.001%, 0.0011%, 0.0012%, 0.0013%, 0.0014%, 0.0015%, 0.002%, 0.003%, 0.005%, 0.01%, 0.05%, 0.1%, 0.15%, 0.2%, 0.5%, 1%, 3%, or ranges including and/or spanning the aforementioned values.

As disclosed elsewhere herein, in several embodiments, the skin healing formulation may include one or more emollient. In several embodiments, the emollient is selected from the group consisting of butylene glycol, caprylic triglyceride, capric triglyceride, and any combination of the foregoing. In several embodiments, the emollient (or emollients), collectively or individually, are present in the skin healing formulation at a weight percent of equal to or less than about: 0.1%, 0.5%, 1%, 3%, 4%, 5%, 7.7%, 7.8%, 799%, 8%, 8.1%, 83%, 8.4%, 8.5%, 8.6%, 8.7%, 8.8%, 9%, 10%, 15%, 20%, 25%, 30%, 50%, or ranges including and/or spanning the aforementioned values.

In several embodiments, as disclosed elsewhere herein, the skin healing formulation comprises butylene glycol. In several embodiments, the butylene glycol is present in the skin healing formulation at a weight percent of equal to or less than about: 0.1%, 0.5%, 1%, 3%, 3.3%, 3.4%, 3.5%, 3.6%, 3.7%, 3.8%, 4%, 5%, 8%, 9%, 10%, 15%, 20%, 25%, 30%, or ranges including and/or spanning the aforementioned values.

In several embodiments, as disclosed elsewhere herein, the skin healing formulation comprises caprylic triglyceride. In several embodiments, the caprylic triglyceride is present in the skin healing formulation at a weight percent of equal to or less than about: 0.1%, 0.5%, 1%, 2%, 2.1%, 2.2%, 2.3%, 2.4%, 2.5%, 2.6%, 2.7%, 2.8%, 2.9%, 3%, 4%, 5%, 5.5%, 6%, 7% 8%, 9%, 10%, 15%, 20%, 25%, 30%, or ranges including and/or spanning the aforementioned values.

In several embodiments, as disclosed elsewhere herein, the skin healing formulation comprises capric triglyceride. In several embodiments, the capric triglyceride is present in the skin healing formulation at a weight percent of equal to or less than about: 0.1%, 0.5%, 1%, 2%, 2.1%, 2.2%, 2.3%, 2.4%, 2.5%, 2.6%, 217%, 2.8%, 2.9%, 3%, 4%, 5%, 5.5%, 6%, 7% 8%, 9%, 10%, 15%, 20%, 25%, 30%, or ranges including and/or spanning the aforementioned values.

As disclosed elsewhere herein, in several embodiments, the skin healing formulation may include one or more emulsifier. In several embodiments, the emulsifier is polysorbate 80. In several embodiments, the emulsifier (or emulsifiers), collectively or individually, are present in the skin healing formulation at a weight percent of equal to or less than about: 0.01%, 0.5%, 0.8%, 0.85%, 0.9%, 0.95%, 0.96%, 0.97%, 0.98%, 1%, 1.1%, 1.2%, 1.3%, 1.4%, 1.5%, 2%, 5%, 7.5%, 10%, 15%, 20%, 25%, 30%, or ranges including and/or spanning the aforementioned values.

In several embodiments, as disclosed elsewhere herein, the skin healing formulation comprises polysorbate 80. In several embodiments, the polysorbate 80 is present in the skin healing formulation at a weight percent of equal to or less than about: 0.01%, 0.5%, 0.8%, 0.85%, 0.9%, 0.95%, 0.96%, 0.97%, 0.98%, 0.99%, 1%, 1.1%, 1.2%, 1.3%, 1.4%. 1.5%, 2%, 5%, 7.5%, 10%, 15%, 20%, 25%, 30%, or ranges including and/or spanning the aforementioned values.

As disclosed elsewhere herein, in several embodiments, the skin healing formulation may include one or more fragrance source. In several embodiments, the fragrance source is Vibrance Type FW. In several embodiments, the fragrance source (or sources), collectively or individually, are present in the skin healing formulation at a weight percent of equal to or less than about: 0.01%, 0.05%, 008%, 0.09%, 0.1%, 0.11%, 0.12%, 0.13%, 0.14%, 0.15%, 0.16%, 0.165%, 0.17%, 0.175%, 0.18%, 0.19%, 0.2%, 0.5%, 1%, 5%, 10%, 15%, 20%, or ranges including and/or spanning the aforementioned values.

In several embodiments, as disclosed elsewhere herein, the skin healing formulation comprises Vibrance Type FW. In several embodiments, the Vibrance Type FW is present in the skin healing formulation at a weight percent of equal to or less than about: 0.01%, 0.05%, 0.08%, 0.09%, 0.1%, 0.11%, 0.12%, 0.13%, 0.14%, 0.15%, 0.16%, 0.165%, 0.17%, 0.175%, 0.18%, 0.19%, 0.2%, 0.5%, 1%, 5%, 10%, 15%, 20%, or ranges including and/or spanning the aforementioned values.

As disclosed elsewhere herein, in several embodiments, the skin healing formulation may include one or more humectant. In several embodiments, the humectant is selected from the group consisting of glycerine, propylene glycol, propanediol, sodium lactate, sodium pyroglutamic acid, and any combination of the foregoing. In several embodiments, the humectant (or humectants), collectively or individually, are present in the skin healing formulation at a weight percent of equal to or less than about: 0.01%, 0.05%, 0.1%, 0.5%, 1%, 5%, 6%0, 7%, 8%, 8.5%, 9%, 9.1%, 9.2%, 9.3%, 9.4%, 9.5%, 9.6%, 10%, 11%, 12%, 13%, 14%, 15%, 20%, 30%, 50%, or ranges including and/or spanning the aforementioned values.

In several embodiments, as disclosed elsewhere herein, the skin healing formulation comprises glycerine. In several embodiments, the glycerine is present in the skin healing formulation at a weight percent of equal to or less than about: 0.01%, 0.05%, 0.1%, 0.5%, 1%, 5%, 5.5%, 5.8%, 5.9%, 6%, 6.1%, 6.2%, 6.3%, 6.4%, 6.5%, 6.7%, 7%, 8%, 9%, 10%, 15%, 20%, 30%, 50%, or ranges including and/or spanning the aforementioned values.

In several embodiments, as disclosed elsewhere herein, the skin healing formulation comprises propylene glycol. In several embodiments, the propylene glycol is present in the skin healing formulation at a weight percent of equal to or less than about: 0.01%, 0.05%, 0.1%, 0.5%, 1%, 2%, 2.1%, 2.2%, 2.3%, 2.4%, 2.5%, 2.6%, 2.7%, 2.8%, 2.9%, 3%, 5%, 10%, 15%, 20%, 30%, 50%, or ranges including and/or spanning the aforementioned values.

In several embodiments, as disclosed elsewhere herein, the skin healing formulation comprises propanediol. In several embodiments, the propanediol is present in the skin healing formulation at a weight percent of equal to or less than about: 0.001%, 0.005%, 0.01%, 0.05%, 0.1%, 0.2%, 0.21%, 0.22%, 0.23%, 0.24%, 0.25%, 0.26%, 0.27%, 0.28%, 0.29%, 0.39%, 0.32%, 0.35%, 0.5%, 1%, 5%, 10%, 15%, 209%, or ranges including and/or spanning the aforementioned values.

In several embodiments, as disclosed elsewhere herein, the skin healing formulation comprises sodium lactate. In several embodiments, the sodium lactate is present in the skin healing formulation at a weight percent of equal to or less than about: 0.0001%), 0.0005%, 0.001%, 0.005%, 0.01%, 0.05%, 0.055%, 0.057%, 0.058%, 0.059%, 0.06%, 0.061%, 0.062%, 0.063%, 0.064%, 0.065%, 0.066%, 0.08%, 0.1%, 0.2%, 0.5%, 1%, 2%, 3%, or ranges including and/or spanning the aforementioned values.

In several embodiments, as disclosed elsewhere herein, the skin healing formulation comprises sodium pyroglutamic acid. In several embodiments, the sodium pyroglutamic acid is present in the skin healing formulation at a weight percent of equal to or less than about: 0.0001%, 0.0005%, 0.001%, 0.005%, 0.01%, 0.05%, 0.055%, 0.07%, 0.071%, 0.072%, 0.073%, 0.074%, 0.075%, 0.076%, 0.077%, 0.078%, 0.079%, 0.08%, 0.1%, 0.2%, 0.5%, 1%, 2%, 3%, or ranges including and/or spanning the aforementioned values.

As disclosed elsewhere herein, in several embodiments, the skin healing formulation may include one or more moisturizer. In several embodiments, the moisturizer is selected from the group consisting of panthenol, hydrolyzed yeast protein, cholecalciferol, and any combination of the foregoing. In several embodiments, the moisturizer (or moisturizers), collectively or individually, are present in the skin healing formulation at a weight percent of equal to or less than about: 0.001%, 0.01%, 0.05%, 0.07%, 0.09%, 0.1%, 0.15%, 0.2%, 0.4%, 0.47%, 0.48%, 0.49%, 0.5%, 0.51%, 0.52%, 0.53%, 0.54%, 0.55%, 0.6%, 0.8%, 1%, 2%, 3%, 5%, 10%, or ranges including and/or spanning the aforementioned values.

In several embodiments, as disclosed elsewhere herein, the skin healing formulation comprises panthenol. In several embodiments, the panthenol is present in the skin healing formulation at a weight percent of equal to or less than about: 0.001%, 0.01%, 0.05%, 0.07%, 0.09%, 0.1%, 0.15%, 0.2%, 0.4%, 0.47%, 0.48%, 0.49%, 0.5%, 0.51%, 0.52%, 053%, 0.54%, 0.55%, 0.6%, 0.8%, 1%, 2%, 3%, or ranges including and/or spanning the aforementioned values.

In several embodiments, as disclosed elsewhere herein, the skin healing formulation comprises hydrolyzed yeast protein. In several embodiments, the hydrolyzed yeast protein is present in the skin healing formulation at a weight percent of equal to or less than about: 0.0001%, 0.0005%, 0.001%, 0.005%, 0.01%, 0.015%, 0.018%, 0.019%, 0.02%, 0.021%, 0.022%, 0.023%, 0.024%, 0.025%, 0.03%, 0.05%, 0.1%, 0.15%, 0.2%, 0.5%, 0.8%, 1%, 2%, 3%, or ranges including and/or spanning the aforementioned values.

In several embodiments, as disclosed elsewhere herein, the skin healing formulation comprises cholecalciferol. In several embodiments, the cholecalciferol is present in the skin healing formulation at a weight percent of equal to or less than about: 0.0000001%, 0.0000005%, 0.000001%, 0.000005%, 0.0000053%, 0.0000054%, 0.0000055%, 0.0000056%, 0.0000057%, 0.0000058%, 0.0000059%, 0.000006%, 0.00001%, 0.00005%, 0.0001%, 0.0005%, 0.001%, 0.005%, 0.01%, 0.05%, 0.1%, or ranges including and/or spanning the aforementioned values.

As disclosed elsewhere herein, in several embodiments, the skin healing formulation may include one or more rubifacient. In several embodiments, the rubifacient is methyl nicotinate. In several embodiments, the rubifacient (or rubifacients), collectively or individually, are present in the skin healing formulation at a weight percent of equal to or less than about: 0.01%, 0.5%, 0.8%, 0.85%, 0.9%, 0.95%, 0.96%, 0.97%, 0.98%, 1%, 1.1%, 1.2%, 1.3%, 1.4%. 1.5%, 2%, 5%, 7.5%, 10%, 15%, 20%, 25%, 30%, or ranges including and/or spanning the aforementioned values. In several embodiments, the rubifacient is methyl nicotinate.

In several embodiments, as disclosed elsewhere herein, the skin healing formulation comprises methyl nicotinate. In several embodiments, methyl nicotinate is present in the skin healing formulation at a weight percent of equal to or less than about: 0.01%, 0.5%, 0.8%, 0.85%, 0.9%, 0.95%, 0.96%, 0.97%, 0.98%, 1%, 1.1%, 1.2%, 1.3%, 1.4%. 1.5%, 2%, 5%, 75%, 10%, 15%, 20%, 25%, 30%, or ranges including and/or spanning the aforementioned values.

As disclosed elsewhere herein, in several embodiments, the skin healing formulation may include one or more antimicrobial. In several embodiments, the antimicrobial is selected from the group consisting of phenyl propanol, caprylyl glycol, and any combination of the foregoing. In several embodiments, the antimicrobial (or antimicrobials), collectively or individually, are 0.001%, 0.005%, 0.01%, 0.05%, 0.1%, 0.2%, 0.3%, 0.5%, 0.75%, 0.76%, 0.77%, 0.78%, 0.79%, 0.8%, 0.81%, 0.82%, 0.83%, 0.85%, 0.86%, 0.87%, 0.88%, 0.9%, 0.95*%, 1%, 5%, 10%, present in the skin healing formulation at a weight percent of equal to or less than about: or ranges including and/or spanning the aforementioned values.

In several embodiments, as disclosed elsewhere herein, the skin healing formulation comprises phenyl propanol. In several embodiments, the phenyl propanol is present in the skin healing formulation at a weight percent of equal to or less than about: 0.001%, 0.005%, 0.01%, 0.05%, 0.1%, 0.3%, 0.5%, 0.51%, 0.52%, 0.53%, 0.54%, 0.55%, 0.56%, 0.57%, 0.58%, 0.59%, 0.6%, 0.8%, 0.85%, 0.9%, 1%, 1.1%, 1.2%, 1.3%, 1.4%. 1.5%, 2%, 5%, 7.5%, 10%, or ranges including and/or spanning the aforementioned values.

In several embodiments, as disclosed elsewhere herein, the skin healing formulation comprises caprylyl glycol. In several embodiments, the caprylyl glycol is present in the skin healing formulation at a weight percent of equal to or less than about: 0.001%, 0.005%, 0.01%, 0.05%, 0.1%, 0.15%, 0.2%, 0.25%, 0.26%, 0.27%, 0.275%, 0.278%, 0.28%, 0.282%, 0.285%, 0.29%, 0.3%, 0.4%, 0.5%, 0.6%, 0.8%, 0.9%, 1%, 1.5%, 2% 5%, 10%, or ranges including and/or spanning the aforementioned values.

As disclosed elsewhere herein, in several embodiments, the skin healing formulation is water based. In several embodiments, water is present in the skin healing formulation at a weight percent of equal to or less than about: 40%, 45%, 50%, 55%, 60%, 65%, 70%, 75%, 80%, 85%, 90%, or ranges including and/or spanning the aforementioned values.

In several embodiments, a subgroup of the components includes troxerutin, escin, ruscus aculeatus root, and acacia senegal gum. In several embodiments, the sum of the components of the subgroup adds up to 100 wt %. In several embodiments, troxerutin is present in the subgroup at a weight percent of equal to or less than about: 10%, 20%, 30%, 50%, 60%, 64%, 65%, 66%, 67%, 68%, 69% 70%, 71%, 72%, 73%, 75%, 80%, 85%, 90%, 95%, 98%, or ranges including and/or spanning the aforementioned values. In several embodiments, escin is present in the subgroup at a weight percent of equal to or less than about: 0.5%, 1%, 3%, 6%, 9%, 10%, 11%, 12%, 13%, 15%, 18%, 20%, 25%, 30%, 35% 40%, or ranges including and/or spanning the aforementioned values. In several embodiments, ruscus aculeatus root is present in the subgroup at a weight percent of equal to or less than about: 0.3%, 0.5%, 1%, 3%, 3.5%, 4%, 4.5%, 5%, 5.5%, 6%, 6.5%, 7%, 9%, 10%, 11%, 12%, 13%, 15%, 18%, 20%, or ranges including and/or spanning the aforementioned values. In several embodiments, acacia senegal gum is present in the subgroup at a weight percent of equal to or less than about: 2%, 3%, 6%, 9%, 10%, 11%, 12%, 13%, 14%, 15%, 16%, 17%, 18%, 19%, 20%, 25%, 30%, 35%, 40%, or ranges including and/or spanning the aforementioned values.

In several embodiments, based on a total weight of the skin healing formulation, troxerutin is present at a weight percent from about 0.95% to about 1.05%; Ginkgo biloba is present at a weight percent from about 0.19% to about 0.21%; escin is present at a weight percent from about 0.14% to about 0.16%; ruscus aculeatus root is present at a weight percent from about 0.074% to about 0.0760%; allantoin is present at a weight percent from about 0.38% to about 0.42%; ammonium glycyrrhizate is present at a weight percent from about 0.048% to about 0.052%; acacia senegal gum is present at a weight percent from about 0.24% to about 0.26%; xanthan gum is present at a weight percent from about 0.20% to about 0.22%; Calendula officinalis is present at a weight percent from about 0.017% to about 0.018%; centella asiatica is present at a weight percent from about 0.032% to about 0.033%; arginine is present at a weight percent from about 0.038% to about 0.042%; aspartic acid is present at a weight percent from about 0.025% to about 0.027%; pyroglutamic acid is present at a weight percent from about 0.020% to about 0.022%; glycine is present at a weight percent from about 0.0061% to about 0.0067%; alanine is present at a weight percent from about 0.0058% to about 0.0062%; serine is present at a weight percent from about 0.0038% to about 0.0042%; valine is present at a weight percent from about 0.0031% to about 0.0033%; proline is present at a weight percent from about 0.0019% to about 0.0021%; threonine is present at a weight percent from about 0.0019% to about 0.0021%; isoleucine is present at a weight percent from about 0.0019% to about 0.0021%; histidine is present at a weight percent from about 0.00076% to about 0.00084%; phenylalanine is present at a weight percent from about 0.00076% to about 0.00084%; tetrahexyldecyl ascorbate is present at a weight percent from about 0.010% to about 0,012%; tocopherol is present at a weight percent from about 0.001% to about 0.0012%; butylene glycol is present at a weight percent from about 3.3% to about 3.7%; caprylic triglyceride is present at a weight percent from about 2.4% to about 2.6%; capric triglyceride is present at a weight percent from about 2.4% to about 2.6%; polysorbate 80 is present at a weight percent from about 0.95% to about 1.05%; Vibrance Type FW is present at a weight percent from about 0.16% to about 0.18%; glycerin is present at a weight percent from about 6.1% to about 6.7%; propylene glycol is present at a weight percent from about 2.4% to about 2.6%; propanediol is present at a weight percent from about 0.26% to about 0.28%; sodium lactate is present at a weight percent from about 0.057% to about 0.063%; sodium pyroglutamic acid is present at a weight percent from about 0.071% to about 0.079%; panthenol is present at a weight percent from about 0.48% to about 0.52%; hydrolyzed yeast protein is present at a weight percent from about 0.019% to about 0.021%; cholecalciferol is present at a weight percent from about 0.0000056% to about 0.000006%; methyl nicotinate is present at a weight percent from about 1.05% to about 1.15%; phenyl propanol is present at a weight percent from about 0.5% to about 0.6%; caprylyl glycol is present at a weight percent from about 0.26% to about 0.3%; and water is present at a weight percent from about 71.8% to about 86.8%.

The skin healing formulation disclosed herein may be prepared by methods described herein, or by modification of these methods. Ways of modifying the methodology include, among others, temperature, solvent, reagents etc., known to those skilled in the art. The skin healing formulations can be prepared using standard pharmaceutical formulation techniques, such as those disclosed in Remington's The Science and Practice of Pharmacy, 21st Ed., Lippincott Williams & Wilkins (2005), incorporated by reference in its entirety.

The skin healing formulations useful as described herein may be formulated for topical use as creams, ointments, gels, solutions or suspensions, etc., containing the ingredients disclosed herein. Topical formulations may generally be comprised of a pharmaceutical carrier, co-solvent, emulsifier, penetration enhancer, preservative system, and emollient in any of a variety of suitable forms for a variety of routes for administration, for example, topical (including transdermal), nasal, rectal, intracerebral, intracranial, intrathecal, intra-arterial, intravenous, intramuscular, subcutaneous, or other parental routes of administration. In some embodiments, the compositions may be in a form suitable for subcutaneous administration. The skilled artisan will appreciate that nasal compositions comprise compositions that are administered by inhalation, and made using available methodologies. Depending upon the particular route of administration desired, a variety of pharmaceutically-acceptable carriers well-known in the art may be used. The term “pharmaceutically acceptable carrier” or “pharmaceutically acceptable excipient” includes any and all solvents, dispersion media, coatings, antibacterial and antifungal agents, isotonic and absorption delaying agents and the like. Pharmaceutically-acceptable carriers include, for example, solid or liquid fillers, diluents, hydrotropies, surface-active agents, and encapsulating substances. Techniques and compositions for making formulations useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In addition to the selected ingredients as described above, some embodiments include formulations containing various adjuvants such as are commonly used in the art may be included. Considerations for the inclusion of various components in skin healing formulations are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions. Preservatives that may be used in the pharmaceutical compositions disclosed herein include, but are not limited to, benzalkonium chloride, P11 MB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

In several embodiments, the skin healing formulation is applied topically to the skin of a subject in need thereof. In several embodiments, the subject is a mammal. In several embodiments, the subject is a human.

In several embodiments, the skin healing formulation is configured to be applied as a roll-on formulation.

### Methods of Treatment

Some embodiments pertain to a method of ameliorating or treating a vascular network disorder of a subject in need thereof. In several embodiments, the method further comprises treating varicose veins. In several embodiments, the method comprises treating a symptom of varicose veins. In several embodiments, the method comprises improving the appearance of varicose veins and/or reducing the prominence of varicose veins of the subject. In several embodiments, the method further comprises alleviating or reducing pain of varicose veins of the subject.

Some embodiments pertain to a method of ameliorating or treating pain of a subject in need thereof. In several embodiments, the method further comprises reducing or lessening the pain of the subject. In several embodiments, the method further comprises reducing or lessening the pain caused by nerve cells and/or neurons (e.g., sciatic nerve), of the subject. In several embodiments, the method further comprises treating the extremities of the subject to reduce or lessen the pain of the subject. In several embodiments, the method further comprises treating the extremities of the subject to reduce or lessen the pain caused by nerve cells and/or neurons (e.g., sciatic nerve), of the subject.

In several embodiments, the method comprises administering to the subject a skin healing formulation (e.g., a therapeutically effective amount). In several embodiments, the skin healing formulation is applied to a portion of skin to be treated (e.g., having pain and/or varicose veins, etc.). In several embodiments, the skin healing formulation is applied to an appendage of the subject. In several embodiments, the skin healing formulation is applied to a leg of the patient. In several embodiments, the skin healing formulation is applied to the lower leg of the patient. In several embodiments, the skin healing formulation is applied to the posterior portion of the leg. In several embodiments, the skin healing formulation is applied as a roll-on.

In several embodiments, after an initial application to an area to be treated (e.g., an area of the subject's skin), additional applications can be made to that application at various time intervals. For example, a first application could be applied and a second application could be applied 10 minutes later. In several embodiments, the method comprises one or more applications (2, 3, 4, 5, 6, or more) to an area to be treated. In several embodiments, where a plurality of applications are used to apply the skin healing formulation, the applications may be spaced by intervals of equal to or less than about: 10 minutes, 15 minutes, 20 minutes, 30 minutes, an hour, two hours, or ranges including and/or spanning the aforementioned values.

As disclosed elsewhere herein, in several embodiments, a therapeutically effective amount of skin healing formulation is applied an area of skin. In several embodiments, an effective amount the skin healing formulation may comprise a weight of the formulation equal to or less than about: 0.25 g, 0.5 g, 1 g, 2 g, 3 g, 5 g, or ranges including and/or spanning the aforementioned values. In several embodiments, an effective amount of skin healing formulation is applied to an area of skin equal to or less than about: 1 square inch, 2 square inches, 4 square inches, 8 square inches, or ranges including and/or spanning the aforementioned values.

In several embodiments, the skin healing formulations provide surprisingly improved results. For instance, in several embodiments, the disclosed skin healing formulations have improved time of onset of efficacy, improved duration of efficacy, and/or improved treatment results (e.g., improved reduction of symptoms). In several embodiments, improved results associated with the disclosed skin healing formulations (e.g., efficacy) can refer to a reduction in a condition (e.g., varicose veins) or symptom of a condition (e.g., pain, appearance, coloration, size of a vein, elevation of a vein from surrounding skin, etc.). In several embodiments, efficacy is measured as a reduction of a condition or a symptom (e.g., a symptom of a condition) by equal to or at least about: 30%, 35%, 40%, 45%, 50%, 55%, 60%, 65%, 70%, 75%, 80%, 85%, 90%, 95%, 100%, or ranges spanning and/or including the aforementioned values.

In several embodiments, measures of efficacy can be qualitative (e.g., an estimate based on a subject's perception through touch or sight such as visibly reduced varicose veins (e.g., smooth or substantially smooth skin)) and/or quantitative (measurement of, for example, the height of a varicose vein from the skin). In several embodiments, severity of a symptom may be measured using a subjective scale based on a patient's perception (e.g., a rating from 1 to 10 of the pain associated with and/or appearance of a varicose vein).

In several embodiments, symptoms of varicose veins may include visible, blue, and/or enlarged veins in your legs. In several embodiments, symptoms of varicose veins may include aching pain (especially after long periods of standing or sitting). In several embodiments, symptoms of varicose veins may include throbbing or cramping in the thigh or calf. In several embodiments, symptoms of varicose veins may include a feeling of heaviness in the leg(s). In several embodiments, symptoms of varicose veins may include swelling in the lower leg, ankle, or foot. In several embodiments, symptoms of varicose veins may include itching in the affected limb.

In several embodiments, the disclosed skin healing formulations provided improved results relative to comparator varicose vein treating topical formulations. In several embodiments, the skin healing formulation provides efficacy that is improved relative to a comparator formulation by equal to or at least about 30%, 35%, 40%, 45%, 50%, 55%, 60%, 65%, 70%, 75%, 80%, 85%, 90%, 95%, 100%, or ranges spanning and/or including the aforementioned values.

In several embodiments, after application, the skin healing formulation has a rapid onset of efficacy. In several embodiments, after application, the skin healing formulation reaches equal to or at least about 50%, 80%, 90%, or 100% of its peak efficacy in a period of less than or equal to about: 2.5 minutes, 5 minutes, 10 minutes, 15 minutes, 20 minutes, 30 minutes, 40 minutes, 50 minutes, 1 hour, 1.5 hours, or ranges including and/or spanning the aforementioned values. In several embodiments, after application, the skin healing formulation has a long duration of efficacy. In several embodiments, the skin healing formulation maintains equal to or at least about 50%, 80%, 90%, or 100% of its peak efficacy for a duration of equal to or at least about: 2.5 hours, 3 hours, 4 hours, 5 hours, 6 hours, 7 hours, 8 hours, 10 hours, 12 hours, 18 hours, 24 hours, 36 hours, or ranges including and/or spanning the aforementioned values.

In several embodiments, the skin healing formulation used in the method is as disclosed anywhere else herein. For example, in several embodiments, the skin healing formulation comprises a) at least one bioflavonoid, b) at least one saponin, and c) at least one skin soothing agent. In several embodiments, the at least one bioflavonoid is troxerutin. In several embodiments, the at least one saponin is selected from the group consisting of allantoin, ammonium glycyrrhizate, escin, ruscus aculeatus root, and any combination of the foregoing. In several embodiments, the at least one skin soothing agent is acacia senegal gum.

In several embodiments of the method, the formulation further comprises at least one active botanical compound. In several embodiments, the at least one active botanical compound is selected from the group consisting of Calendula officinalis, Centella sciatica, and any combination of the foregoing.

In several embodiments of the method, the skin healing formulation further comprises at least one amino acid selected from the group consisting of arginine, aspartic acid, pyroglutamic acid, glycine, alanine, serine, valine, proline, threonine, isoleucine, histidine, phenylalanine, and any combination of the foregoing. In several embodiments, the skin healing formulation further comprises at least one antioxidant selected from the group consisting of tetrahexyldecyl ascorbate, tocopherol and any combination of the foregoing. In several embodiments, the skin healing formulation further comprises at least one emollient selected from the group consisting of butylene glycol, caprylic triglyceride, capric triglyceride, and any combination of the foregoing. In several embodiments, the skin healing formulation further comprises at least one emulsifier wherein the at least one emulsifier is polysorbate 80. In several embodiments, the skin healing formulation further comprises at least one fragrance source wherein the at least one fragrance source is Vibrance Type FW. In several embodiments, the skin healing formulation further comprises at least one humectant selected from the group consisting of glycerine, propylene glycol, propanediol, sodium lactate, sodium pyroglutamic acid, and any combination of the foregoing. In several embodiments, the skin healing formulation further comprises at least one moisturizer selected from the group consisting of panthenol, hydrolyzed yeast protein, cholecalciferol, and any combination of the foregoing. In several embodiments, the skin healing formulation further comprises at least one rubifacient wherein the at least one rubifacient is methyl nicotinate. In several embodiments, the skin healing formulation further comprises at least one antimicrobial selected from the group consisting of phenyl propanol, caprylyl glycol and any combination of the foregoing.

In several embodiments, the formulation is administered topically. In several embodiments, the formulation comprises a roll-on formulation.

In several embodiments, a delay time after the formulation is administered that the improved appearance begins is equal to or less than about 10 seconds to about 10 minutes. In several embodiments, a time span after the formulation is administered that the improved appearance ends is equal to or more than about 30 minutes to about 8 hours.

In several embodiments, the subject is a mammal. In several embodiments, the subject is a human.

In some embodiments, the skin healing formulation may have positive effect on the skin of the subject. For example, the formulation may improve or restore the balance of skin's microbiome. As a result, the skin's barrier may be properly maintained, and leads to increasing hydration and/or reducing water loss.

Some embodiments include co-administering the skin healing formulation described herein, with an additional medicament. By “co-administration,” it is meant that the skin healing formulation and the medicament may be found in the patient's bloodstream at the same time, regardless of when or how they are actually administered. In one embodiment, the skin healing formulation and the medicament are administered simultaneously. In another embodiment, the skin healing formulation and the medicament are administered sequentially. In one embodiment the agents are administered through the same route, such as topically. In another embodiment, the agents are administered through different routes, such as one being administered topically, another being administered subcutaneously and another being administered iv.

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### EXAMPLES

### Example 1: Raw Material Ingredients

The raw material ingredients used to prepare a skin healing formulation as disclosed herein are listed in Table 1.

TABLE 1

| Commercial | Ingredient | | Component |
|---|---|---|---|
| Source | (wt %) | Component | (wt %) |
| In-House | 75.2669 | Water | 100.0000 |
| PT Ecogreen | 5.5000 | Glycerin | 100.0000 |
| PT Musim Mas | 5.0000 | Caprylic/Capric | 100.0000 |
| BASF | 5.0000 | WaterTriglyceride | 11.1000 |
| | | Butylene Glycol | 70.0000 |
| | | Panthenol | 10.0000 |
| | | Escin | 3.0000 |
| | | Glycerin | 2.0000 |
| | | Ruscus Aculeatus | 1.5000 |
| | | Root Extract | |
| | | Ammonium Glycyrrhizate | 1.0000 |
| | | Centella Asiatica Extract | 0.6500 |
| | | Hydrolyzed Yeast Protein | 0.4000 |
| | | Calendula Officinalis | 0.3500 |
| | | Flower Extract | |
| DOW | 2.5000 | Propylene glycol | 100.0000 |
| Schulke | 1.1000 | Phenyl propanol | 50.0000 |
| | | Caprylyl glycol | 25.0000 |
| | | Propanediol | 24.9000 |
| | | Tocopherol | 0.1000 |
| Caribbean | 1.1000 | Methyl Nicotinate | 100.0000 |
| EMD | 1.000 | Troxerutin | 100.0000 |
| Coast | 1.000 | Polysorbate 80 | 100.0000 |
| Southwest | | | |
| Mel-Co | 1.000 | Glycerin | 80.0000 |
| | | Ginkgo Biloba Leaf Extract | 20.0000 |
| Ajinomoto | 0.5000 | Sodium pyroglutamic acid | 15.0000 |
| | | Sodium Lactate | 12.0000 |
| | | Arginine | 8.0000 |
| | | Aspartic Acid | 5.2000 |
| | | Pyroglutamic acid | 4.2700 |
| | | Glycine | 1.2800 |
| | | Alanine | 1.2000 |
| | | Serine | 0.8000 |
| | | Valine | 0.6400 |
| | | Proline | 0.4000 |
| | | Threonine | 0.4000 |
| | | Isoleucine | 0.4000 |
| | | Histidine | 0.1600 |
| | | Phenylalanine | 0.1600 |
| | | Water | 50.0900 |
| Seppic | 0.4500 | Acacia Senegal Gum | 55.0000 |
| | | Xanthan Gum | 45.0000 |
| Salicy lates | 0.4000 | Allantoin | 100.0000 |
| Agilex | 0.1700 | Fragrance | 100.0000 |
| Barnet | 0.0110 | Tetrahexyldecyl Ascorbate | 100.0000 |
| Products | | | |
| Nutralliance | 0.0020 | Cholecalciferol | 0.2900 |
| | | Acacia Senegal Gum | 99.7100 |
| Corbion | 0.0001 | Lactic Acid | 88.0000 |
| | | Water | 12.0000 |

### Example 2: Raw Materials Testing

The raw material ingredients to be tested under occlusive conditions were placed on an 8-millimeter aluminum Finn Chamber® (Epitest Ltd. Oy, Tuusula, Finland) supported on Scanpor® Tape (Norgesplaster A/S, Kristiansand, Norway) or an 8-millimeter filter paper coated aluminum Finn Chamber® AQUA supported on a thin flexible transparent polyurethane rectangular film coated on one side with a medical grade acrylic adhesive, consistent with adhesive used in state-of-the-art hypoallergenic surgical tape or a 7 mm IQ-ULTRA® closed cell system which is made of additive-free polyethylene plastic foam with a filter paper incorporated. It is supplied in units of 10 chambers on a hypoallergenic non-woven adhesive tape; the width of the tape is 52 mm and the length is 118 mm or other equivalents. Test materials to be tested under semi-occlusive conditions were placed on a test strip with a Rayon/Polypropylene pad or on a 7.5 mm filter paper disc affixed to a strip of hypoallergenic tape (Johnson & Johnson 1 inch First Aid Cloth Tape). Test materials to be tested in an open patch were applied and rubbed directly onto the back of the subject.

Approximately 0.02-0.05 mL for liquid materials and/or 0.02-0.05 g for solid materials of the test material was used for the study. Liquid test material was dispensed on a 7.5 mm paper disk, which fit in the Finn Chamber. Subjects were requested to bathe or wash as usual before arrival at the facility. Patches containing the test material were then affixed directly to the skin of the intrascapular regions of the back, to the right or left of the midline and subjects were dismissed with instructions not to wet or expose the test area to direct sunlight. Patches remained in place for 48 hours after the first application. Subjects were instructed not to remove the patches prior to their 48 hour scheduled visit. Thereafter, subjects were instructed to remove patches 24 hours after application for the remainder of the study. This procedure was repeated until a series of nine (9) consecutive, 24-hour exposures had been made three (3) times a week for three (3) consecutive weeks. Prior to each reapplication, the test sites evaluated by trained laboratory personnel. Following a 10-14 day rest period a retest/challenge dose was applied once to a previously unexposed test site. Test sites were evaluated by trained laboratory personnel 48 and 96 hours after application. In the event of an adverse reaction, the area of erythema and edema were measured. Edema is estimated by the evaluation of the skin with respect to the contour of the unaffected normal skin. Subjects were instructed to report any delayed reactions that might occur after the final reading. Clients were be notified immediately in the case of an adverse reaction and a determination is made as to treatment program if necessary.

### Example 3: Preparation of Skin Healing Formulation

A batch is prepared by combining Sallitoin (0.40 g), Prodew® 500 (0.50 g), and water (57.77 g) with vortex stirring for 15-20 minutes. Puraguard™ (250 g), Ecocerol (5.50 g), and Solagum™ AX (0.45 g) are combined with stirring to form a slurry that is added to the batch. The batch undergoes vortex stirring for 15-20 minutes until smooth and is heated to 45° C. Polysorbate 80 (1.00 g), Vitamin D3 (0.002 g), and MASESTER E6000 (5.00 g) are combined and added to the batch at 45° C. then the batch is mixed with vortex stirring and heated to 65° C. Ginkgo biloba G (1.00 g) and water (17.50 g) are combined and added to the batch at 65° C. then the batch is mixed with vortex stirring for 25-30 minutes and cooled to 40° C. Sensiva PA 40 (1.10 g), methyl nicotinate (1.10 g), RonaCare® troxerutin (1.00 g), Biophytex® LS 9832 (5.00 g), Vibrance Type FW (0.17 g), are BV-OSC (0.011 g) are combined and added to the batch at 40° C. then the batch is mixed for 15-20 minutes with sweep and vortex stirring until smooth. Purac® HS 88 (88% lactic acid) is added to adjust the pH of the batch to a range from 4.00 to 5.00. A quantity of 100.0 g of the skin healing formulation disclosed herein is obtained.

### Example 4: Testing the Skin Healing Formulation

The skin healing formulation prepared in Example 3 was tested using the occlusive test conditions described in Example 2. There were 53 participants enrolled and 51 of the participants completed the study. The study participants were not under a doctor's care and were free of any dermatological or systemic disorder or acute or chronic disease. The study participants ranged in age from 19-64 with 17 males and 34 females. The distribution of Fitzpatrick Skin Types included 3 participants with skin type 2, 28 participants with skin type 3, 18 participants with skin type 4, and 2 participants with skin type 5. The Fitzpatrick Skin Types are: 1—always burn, does not tan; 2—burn easily, tan slightly; 3—burn moderately, tan progressively; 4—burn a little, always tan; 5—rarely burn, tan intensely; 6—never burn, tan very intensely, (Agache, P. et al, Measuring the Skin, (p. 473, Table 48.1) Springer-Verlag Berlin Heidelberg, 2004). Scoring scale and definition of symbols are based on the scoring scheme according to the International Contact Dermatitis Research Group scoring scale (Rietschel, R. L et al, Fisher's Contact Dermatitis, 4^{th} edition, Baltimore, Williams & Wilkins, 1995): 0=no reaction. (negative); 1=erythema throughout at least ¾ of patch area; 2=erythema and induration throughout at least ¾ of patch area; 3=erythema, induration and vesicles; 4=erythema, induration and bullae; D=site discontinued; Dc=subject discontinued voluntarily; and DcI=subject discontinued per investigator. Clinical evaluations were performed by an investigator or designee trained in the clinical evaluation of the skin. Whenever feasible, the same individual did the scoring of all the subjects throughout the study and was blinded to the treatment assignments and any previous scores. Results of the study are displayed in Table 2. No adverse reactions of any kind were reported during the course of this study. There was one subject with a Grade 4 reaction and ten subjects with a Grade 1 reaction to the positive control (2.0% Sodium Lauryl Sulfate Solution). No subjects showed any signs of reaction to the negative control (DI Water).

TABLE 2

| Participant | | | | | | | | | | | | | | |
|---|---|---|---|---|---|---|---|---|---|---|---|---|---|---|
| | | | Skin | Induction | | | | | | | | | Challenge | |
| No. | Sex | Age | Type | 1 | 2 | 3 | 4 | 5 | 6 | 7 | 8 | 9 | 1 | 2 |
| 1 | F | 41 | 4 | 0 | 0 | 0 | 0 | 0 | 0 | 0 | 0 | 0 | 0 | 0 |
| 2 | M | 48 | 4 | 0 | 0 | 0 | 0 | 0 | 0 | 0 | 0 | 0 | 0 | 0 |
| 3 | F | 24 | 3 | 0 | 0 | 0 | 0 | 0 | 0 | 0 | 0 | 0 | 0 | 0 |
| 4 | F | 46 | 3 | 0 | 0 | 0 | 0 | 0 | 0 | 0 | 0 | 0 | 0 | 0 |
| 5 | F | 36 | 4 | Dc | Dc | Dc | Dc | Dc | Dc | Dc | Dc | Dc | Dc | Dc |
| 6 | F | 40 | 4 | 0 | 0 | 0 | 0 | 0 | 0 | 0 | 0 | 0 | 0 | 0 |
| 7 | F | 35 | 3 | 0 | 0 | 0 | 0 | 0 | 0 | 0 | 0 | 0 | 0 | 0 |
| 8 | F | 64 | 3 | 0 | 0 | 0 | 0 | 0 | 0 | 0 | 0 | 0 | 0 | 0 |
| 9 | F | 47 | 3 | 0 | 0 | 0 | 0 | 0 | 0 | 0 | 0 | 0 | 0 | 0 |
| 10 | F | 49 | 4 | 0 | 0 | 0 | 0 | 0 | 0 | 0 | 0 | 0 | 0 | 0 |
| 11 | F | 34 | 3 | 0 | 0 | 0 | 0 | 0 | 0 | 0 | 0 | 0 | 0 | 0 |
| 12 | F | 58 | 2 | 0 | 0 | 0 | 0 | 0 | 0 | 0 | 0 | 0 | 0 | 0 |
| 13 | F | 28 | 3 | 0 | 0 | 0 | 0 | 0 | 0 | 0 | 0 | 0 | 0 | 0 |
| 14 | F | 49 | 3 | 0 | 0 | 0 | 0 | 0 | 0 | 0 | 0 | 0 | 0 | 0 |
| 15 | F | 31 | 3 | 0 | 0 | 0 | 0 | 0 | 0 | 0 | 0 | 0 | 0 | 0 |
| 16 | F | 55 | 3 | 0 | 0 | 0 | 0 | 0 | 0 | 0 | 0 | 0 | 0 | 0 |
| 17 | F | 21 | 3 | 0 | 0 | 0 | 0 | 0 | 0 | 0 | 0 | 0 | 0 | 0 |
| 18 | F | 63 | 4 | 0 | 0 | 0 | 0 | 0 | 0 | 0 | 0 | 0 | 0 | 0 |
| 19 | F | 55 | 4 | 0 | 0 | 0 | 0 | 0 | 0 | 0 | 0 | 0 | 0 | 0 |
| 20 | F | 43 | 4 | 0 | 0 | 0 | 0 | 0 | 0 | 0 | 0 | 0 | 0 | 0 |
| 21 | F | 31 | 3 | 0 | 0 | 0 | 0 | 0 | 0 | 0 | 0 | 0 | 0 | 0 |
| 22 | M | 23 | 3 | 0 | 0 | 0 | 0 | 0 | 0 | 0 | 0 | 0 | 0 | 0 |
| 23 | M | 47 | 4 | 0 | 0 | 0 | 0 | 0 | 0 | 0 | 0 | 0 | 0 | 0 |
| 24 | F | 60 | 3 | 0 | 0 | 0 | 0 | 0 | 0 | 0 | 0 | 0 | 0 | 0 |
| 25 | F | 19 | 3 | 0 | 2 | Dcl | Dcl | Dcl | Dcl | Dcl | Dcl | Dcl | Dcl | Dcl |
| 26 | F | 30 | 2 | 0 | 0 | 0 | 0 | 0 | 0 | 0 | 0 | 0 | 0 | 0 |
| 27 | M | 30 | 4 | 0 | 0 | 0 | 0 | 0 | 0 | 0 | 0 | 0 | 0 | 0 |
| 28 | M | 30 | 4 | 0 | 0 | 0 | 0 | 0 | 0 | 0 | 0 | 0 | 0 | 0 |
| 29 | M | 42 | 3 | 0 | 0 | 0 | 0 | 0 | 0 | 0 | 0 | 0 | 0 | 0 |
| 30 | F | 21 | 4 | 0 | 0 | 0 | 0 | 0 | 0 | 0 | 0 | 0 | 0 | 0 |
| 31 | M | 45 | 5 | 0 | 0 | 0 | 0 | 0 | 0 | 0 | 0 | 0 | 0 | 0 |
| 32 | F | 20 | 4 | 0 | 0 | 0 | 0 | 0 | 0 | 0 | 0 | 0 | 0 | 0 |
| 33 | F | 19 | 4 | 0 | 0 | 0 | 0 | 0 | 0 | 0 | 0 | 0 | 0 | 0 |
| 34 | M | 24 | 3 | 0 | 0 | 0 | 0 | 0 | 0 | 0 | 0 | 0 | 0 | 0 |
| 35 | F | 25 | 4 | 0 | 0 | 0 | 0 | 0 | 0 | 0 | 0 | 0 | 0 | 0 |
| 36 | F | 21 | 3 | 0 | 0 | 0 | 0 | 0 | 0 | 0 | 0 | 0 | 0 | 0 |
| 37 | M | 19 | 3 | 0 | 0 | 0 | 0 | 0 | 0 | 0 | 0 | 0 | 0 | 0 |
| 38 | M | 41 | 4 | 0 | 0 | 0 | 0 | 0 | 0 | 0 | 0 | 0 | 0 | 0 |
| 39 | F | 30 | 3 | 0 | 0 | 0 | 0 | 0 | 0 | 0 | 0 | 0 | 0 | 0 |
| 40 | F | 46 | 4 | 0 | 0 | 0 | 0 | 0 | 0 | 0 | 0 | 0 | 0 | 0 |
| 41 | F | 38 | 4 | 0 | 0 | 0 | 0 | 0 | 0 | 0 | 0 | 0 | 0 | 0 |
| 42 | M | 39 | 3 | 0 | 0 | 0 | 0 | 0 | 0 | 0 | 0 | 0 | 0 | 0 |
| 43 | F | 33 | 3 | 0 | 0 | 0 | 0 | 0 | 0 | 0 | 0 | 0 | 0 | 0 |
| 44 | F | 28 | 3 | 0 | 0 | 0 | 0 | 0 | 0 | 0 | 0 | 0 | 0 | 0 |
| 45 | M | 49 | 3 | 0 | 0 | 0 | 0 | 0 | 0 | 0 | 0 | 0 | 0 | 0 |
| 46 | F | 46 | 2 | 0 | 0 | 0 | 0 | 0 | 0 | 0 | 0 | 0 | 0 | 0 |
| 47 | M | 39 | 4 | 0 | 0 | 0 | 0 | 0 | 0 | 0 | 0 | 0 | 0 | 0 |
| 48 | F | 36 | 3 | 0 | 0 | 0 | 0 | 0 | 0 | 0 | 0 | 0 | 0 | 0 |
| 49 | M | 37 | 3 | 0 | 0 | 0 | 0 | 0 | 0 | 0 | 0 | 0 | 0 | 0 |
| 50 | M | 27 | 3 | 0 | 0 | 0 | 0 | 0 | 0 | 0 | 0 | 0 | 0 | 0 |
| 51 | M | 45 | 3 | 0 | 0 | 0 | 0 | 0 | 0 | 0 | 0 | 0 | 0 | 0 |
| 52 | M | 38 | 5 | 0 | 0 | 0 | 0 | 0 | 0 | 0 | 0 | 0 | 0 | 0 |
| 53 | F | 30 | 3 | 0 | 0 | 0 | 0 | 0 | 0 | 0 | 0 | 0 | 0 | 0 |

### Example 5: Treatment of Varicose Veins with the Skin Healing Formulation

A roll-on applicator containing the skin healing formulation prepared in Example 3 was used to treat the skin of 35 study participants enrolled in a two week study. The study participants each had varicose veins with half of the participants reporting discomfort or pain and were evaluated for each of the parameters listed in Table 3 before the study began. The participants applied the roll-on applicator down the length of the varicose vein(s), beginning two inches above where the vein(s) begins to bulge or is visible, stopping two inches below where the vein(s) visibly ends. The participants used their fingers to gently massage the vein(s) and spread the product thoroughly throughout the affected area. The skin became warm and red for approximately 15-20 minutes and the formulation was applied a second time after 20 minutes. The formulation was applied twice in the morning and twice in the evening waiting at least 8 hours between applications and was applied twice each day for a time period of two weeks. The participants were evaluated according to the results listed in Table 3.

TABLE 3

| | | Intervals | | | | |
|---|---|---|---|---|---|---|
| | | | | | | 1 or 2 |
| | | | | | | Weeks |
| Claim | Methodology | Baseline | 1 Hour | 8 Hour* | 24 Hour | (Final) |
| Reduction in | Expert grading of | Yes | Yes | NA | Yes | Yes |
| appearance of | digital photographs | Yes | Yes | NA | Yes | Yes |
| varicose veins | Cutometer | | | | | |
| Improves skin firmness | | | | | | |
| and elasticity | Questionnaire** | NA | Yes | Yes | Yes | Yes |
| Subjective | | | | | | |

*At home interval
**Questionnaire is limited to 25 questions per interval

### Example 6: Reduction in Appearance of Varicose Veins with the Skin Healing Formulation

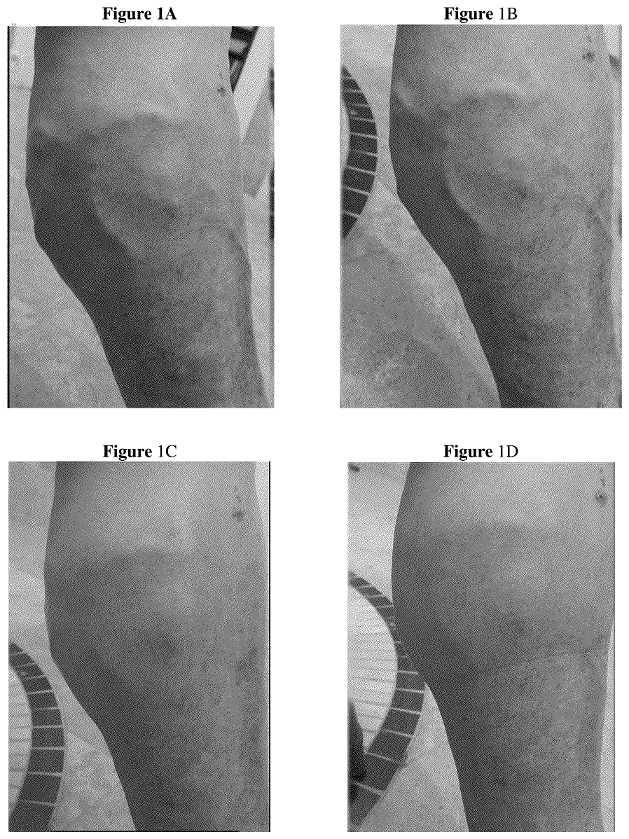

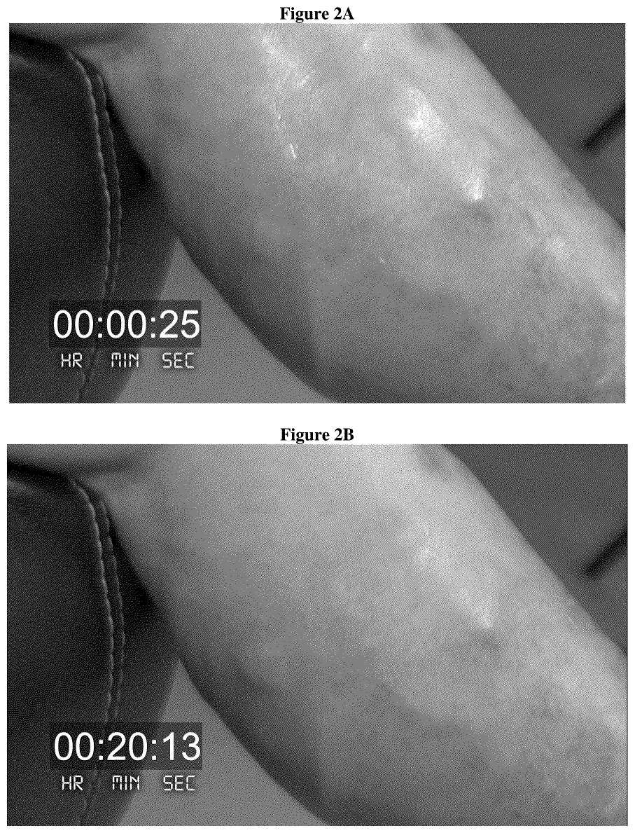

A roll-on applicator containing the skin healing formulation prepared in Example 3 was used to treat the skin of the posterior lower leg of a study participant in a one month study. The study participant had bulging varicose veins that were visible before the study began was shown in FIG. 1A. The participant applied the roll-on applicator to the bulging varicose veins then massaged the veins and spread the product thoroughly as described in Example 5. The appearance of the bulging varicose veins was documented with photographs and decreased slightly 20 minutes after the formulation was applied, as shown in FIG. 1B The appearance of the bulging varicose veins was reduced significantly 1 hour after the formulation was applied, as shown in FIG. 1C. The participant then applied a second treatment of the roll-on applicator to the bulging varicose veins and massaged the veins and spread the product thoroughly as described in Example 5. The appearance of the bulging varicose veins remained reduced significantly 8 hours after the second treatment was applied, as shown in FIG. 1D.

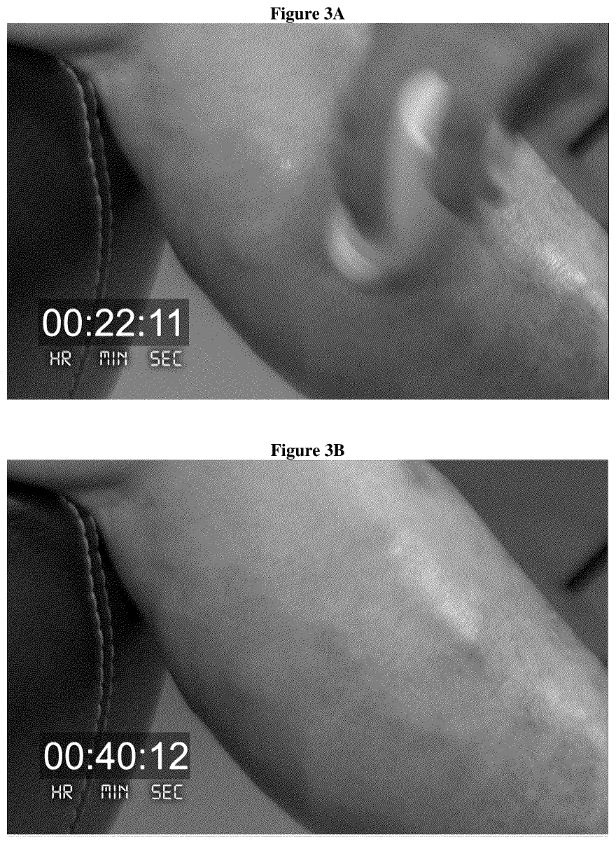

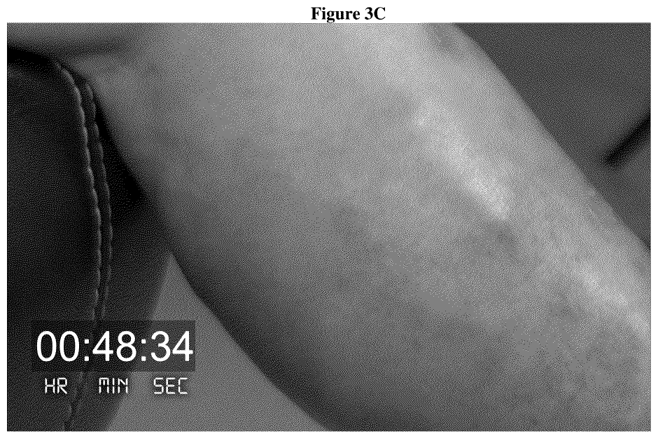

One month later the appearance of the bulging varicose veins was less than it had been at the beginning of the study, as shown in FIG. 2A. The participant then applied a third treatment of the roll-on applicator to the bulging varicose veins and massaged the veins and spread the product thoroughly as described in Example 5. The appearance of the bulging varicose veins was reduced significantly 20 minutes after the third treatment was applied, as shown in FIG. 2B. The participant then applied a fourth treatment of the roll-on applicator to the bulging varicose veins, as shown in FIG. 3A, and massaged the veins and spread the product thoroughly as described in Example 5. The appearance of the bulging varicose veins was nearly imperceptible 18 and 26 minutes after the fourth treatment was applied as shown in FIGS. 3B and 3C, respectively.

### Example 7: Clinical Study of the Skin Healing Formulation

A clinical study of a roll-on applicator containing the skin healing formulation prepared in Example 3 was conducted to evaluate the effectiveness of the skin healing formulation to improve skin firmness and elasticity, to improve appearance of varicose veins, and to improve skin conditions based on consumer perception. The clinical study was conducted using the skin healing formulation that was identified as Varicose Vein Topical, 5% VEIN (ROLL-ON) SERUM #B; Formula Number: C0622-F-39390-004 40 15 Sep. 2021 1172622. The clinical study was conducted over a 4-week period by the BioScreen Clinical Services Division of BioScreen Testing Services, Inc. located at 2300 W. 205th St. Torrance, CA 90501. The roll-on applicator containing the skin healing formulation was applied as described in Example 5 herein. The clinical study was conducted in accordance with the International Conference of Harmonization Tripartite Guideline on Good Clinical Practice, applicable FDA regulations/guidelines set forth in 21 CFR Parts 11, and 50 and standard practices of BioScreen Testing Services. Samples the skin healing formulation were retained for a period of 30 days beyond submission of final report. Sample disposition was conducted in compliance with appropriate federal, state and local ordinances.

A total of 30 healthy subjects consented, enrolled and completed the clinical study as shown in Table 4. Inclusion Criteria: Individuals who, at baseline, are free of any dermatological or systemic disorder, which would interfere with the results, at the discretion of the Investigator. Individuals in good general health. Individuals who complete a preliminary medical history. Individuals who complete a photography release form. Individuals who will read, understand and sign an informed consent document. Individuals who will be able to cooperate with the Investigator and research staff, have the test product applied according to the protocol and complete the full course of the study. Individuals who have not participated in any study involving the same test site (body site where varicose veins are present) for the past 7 days. Individuals with visible varicose veins as determined by BioScreen Clinical Services (BCS) staff. 50% of individuals who report discomfort or pain from the varicose veins. 50% of individuals who do report discomfort or pain from the varicose veins. Individuals who will agree to refrain from using personal care products on their body at the site where varicose veins are present (e.g. lotions and creams) for the washout period and duration of the study, with the exception of those provided by BCS. Individuals who agree not to sunbathe/tan and agree to avoid sun (UV) exposure as much as possible for the duration of the study. Exclusion Criteria: Individuals who have had a history of any acute or chronic disease that could interfere with or increase the risk on study participation. Individuals with an active (flaring) disease or chronic skin allergies (atopic dermatitis/eczema), or had recently treated skin cancer (within the last 12 months). Individuals with damaged skin at or in close proximity to test sites (e.g., sunburn, tattoos, scars, excessive hair, non-removable piercings, or other disfigurations). Individuals with a history of immunosuppression/immune deficiency disorders or currently using immunosuppressive medications (e.g., azathioprine, belimumab, cyclophosphamide, Enbrel, Imuran, Humira, mycophenolate mofetil, methotrexate, prednisone, Remicade, Stelara.) and/or radiation as determined by study documentation. Individuals who have any history, which, in the Investigator's opinion, indicates the potential for harm to the subject or could place the validity of the study in jeopardy. Individuals who indicate that they are pregnant, planning a pregnancy or nursing. Individuals who have been medically diagnosed with Type I Diabetes. Individuals who have a known history of hypersensitivity to any cosmetics, personal care products, and/or fragrances. Individuals who are employees of BCS.

TABLE 4

| No. | Subject ID | Subject Initials | Age | Race | Gender | Pain |
|---|---|---|---|---|---|---|
| 1 | 9918 | M-V | 54 | C | Female | Yes |
| 2 | 10735 | J-F | 62 | AA | Female | No |
| 3 | 14742 | BRB | 52 | AA | Female | Yes |
| 4 | 15586 | NAB | 60 | AA/H | Female | No |
| 5 | 17557 | TNM | 36 | AA | Female | Yes |
| 6 | 18862 | H-T | 60 | A | Female | No |
| 7 | 21767 | MLM | 50 | AA | Female | No |
| 8 | 21917 | C-S | 52 | A | Female | No |
| 9 | 22674 | ADH | 50 | AA | Female | Yes |
| 10 | 22798 | AIM | 51 | A/PI | Female | No |
| 11 | 24464 | JLM | 58 | AA | Female | No |
| 12 | 24588 | BAM | 62 | C | Female | Yes |
| 13 | 24618 | DDH | 63 | A/AA | Female | No |
| 14 | 30016 | YHK | 52 | A | Female | Yes |
| 15 | 32682 | N-T | 51 | A | Female | No |
| 16 | 32779 | PAH | 51 | AA/C | Female | Yes |
| 17 | 33468 | BMB | 65 | C | Female | Yes |
| 18 | 33597 | C-A | 60 | A/PI | Female | No |
| 19 | 33902 | G-T | 51 | C | Female | No |
| 20 | 35927 | DKK | 63 | C | Female | Yes |
| 21 | 36093 | J-C | 47 | H | Female | No |
| 22 | 36230 | BSC | 65 | AA | Female | No |
| 23 | 36724 | CAG | 45 | C | Female | No |
| 24 | 36831 | R-M | 65 | H | Female | Yes |
| 25 | 37951 | ADR | 58 | AA | Female | Yes |
| 26 | 37963 | DTF | 60 | H | Female | No |
| 27 | 38566 | T-S | 43 | C | Female | Yes |
| 28 | 38616 | WRE | 59 | C | Male | Yes |
| 29 | 38655 | ACT | 61 | C | Female | No |
| 30 | 38724 | CHH | 61 | H | Female | No |

AA = African American,
A = Asian,
C = Caucasian,
H = Hispanic,
PI = Pacific Islander

### Bioinstrumental Method to Measure Skin Firmness and Elasticity

The biomechanical properties of human skin are a complex combination of elastic (elastin fibers) and viscous (collagen fibers and surrounding intercellular ground substance) components. The Cutometer allows the measurement of the viscoelastic properties of the skin in vivo. The measuring principle of the Cutometer is based on suction. A defined negative air pressure is created and applied on the skin surface through the opening of a probe drawing the skin into its aperture. The resulting vertical deformation of the skin is measured by determining the depth of skin penetration into the probe. This is achieved by a noncontact optical system consisting of a light transmitter and a light recipient. Two glass prisms project the light from transmitter to recipient, where the diminution of the infrared light beam depending on the penetration depth of the skin is measured. One (1) replicate measurement was taken on each test site at each time point totaling two measurements per time point. References: Undine B, Eisner P., Hardware and Measuring Principle: The Cutometer. In the Bioengineering of the Skin—Skin Biomechanics, 2002, pp. 91-98. Agache P, Varchon D., Skin Mechanical Function. In the Measuring the Skin, 2004, pp. 429-467. Information and operating instructions for the Cutometer MPA 580 and its probes.

### Digital Photography for Expert Grading of Appearance of Varicose Veins

Photographs were taken in accordance with regulations provided by consumer protection agencies such as the Federal Trade Commission, the Food and Drug Administration and several other regulatory authorities. The following guidelines were followed: body position is the same in before and after photos, same lighting conditions are used and the distance from the camera is same for both, before and after picture, and same room and background is used for both before and after picture. Digital photographs were taken of subjects' varicose veins. Photographs obtained were evaluated for the aforementioned parameter using the following scale (half point increments will be allowed): Overall Scale for varicose vein: 0=None, 1-3=Mild, 4-6=Moderate, 7-9=Severe. References: Arch. Dermatol., 1992, 128, pp. 347-351. Br. J. Dermatol., 1994, 130, pp. 167-173. Skin Pharmacol. Appl. Skin Physiol., 2003, 16, pp. 100-107.

### Self Assessment Questionnaire

Each subject was instructed to complete a self-assessment questionnaire at the 1 hour, 8 hour (take-home questionnaire), 24 hour, and 2 week post-treatment intervals.

### Study Protocol

Prospective subjects reported to the facility a minimum of 3 days prior to the start of the study. Prior to beginning all study related activities, prospective subjects completed an informed consent form, medical history form, photography release form, code of conduct form, experimental subject's bill of rights and a HIPAA form. Subjects completed reading and were interviewed to ensure their understanding of the aforementioned forms and were given the opportunity to ask any study related questions. Subjects were enrolled on the basis of the subject selection criteria. Subjects were given specific instructions prohibiting use of all personal care products (i.e., lotions, creams), on the test site for the entire washout and study duration, except for those provided by BCS. Following the washout period subjects returned to the facility for baseline measurements.

Enrolled subjects had the following procedure/measurement performed by trained BCS staff. Baseline (pre-treatment) a. Digital photography b. Skin firmness and elasticity (two) measurements at sites where varicose veins are present. Following baseline measurements, subjects were instructed to use the test product as described in Example 5 herein and to remain in the exam room. At 1 hour (±10 minutes) post-treatment, subjects had the following procedures/measurements a. Digital photography. b. Skin firmness and elasticity (two) measurements at sites where varicose veins are present c. Post-treatment questionnaire. Subjects were dismissed from the testing facility and instructed to return 24 hours (±1 hour) post-application. Subjects were provided with a questionnaire to complete at home at 8 hour (i 30 minutes) post-application. At 24 hours (±1 hour) post-application, subjects had the following procedures/measurements a. Digital photography. b. Skin firmness and elasticity (two) measurements at sites where varicose veins are present. c. Post-treatment questionnaire. Subjects were given the test product to use product as described in Example 5 herein for the duration of the study. Subjects were dismissed from the testing facility and informed to return 2 weeks (±3 days) post-treatment. Test product was weighed for compliance. On the day of their scheduled visit, subjects did not use test product and had the following procedures/measurements: a. Digital photography, b. Skin firmness and elasticity (two) measurements at sites where varicose veins are present. c. Post-treatment questionnaire.

An adverse event is any untoward medical occurrence, whether or not it is considered study related, including death, experienced by a subject. An event may consist of a disease, an exacerbation of a pre-existing illness or condition, an occurrence of an intermittent illness or condition, a set of related symptoms or signs, or a single symptom or sign. No adverse events were reported during the study period.

Statistical analyses tested the hypothesis that the pre-treatment values of each parameter were statistically different from its post-treatment values. Statistical significance was declared if the two-tailed p-value was ≤0.05. The percentage of subjects responding in favor of the test product was reported. Statistical analysis was performed using a z-test. Statistical significance was declared if the p-value is ≤0.05.

### Example 8: Clinical Study Results

### A. Skin Firmness R0 (Cutometer)

A statistically significant change in skin firmness at the immediate post application interval was observed as shown in Table 5. Negative difference indicates an improvement in skin firmness. Individualized skin firmness results are shown in Table 16.

TABLE 5

| Parameter | Immediate | 24 Hour | Week 2 |
|---|---|---|---|
| Mean Percent Difference From | −9.09% | 1.03% | 10.13% |
| Baseline | (p ≤ 0.001) | (NS) | (NS) |
| Percent of Subjects with Change | 30.00% | 46.67% | 43.33% |
| | (NS) | (NS) | (NS) |

### B. Skin Elasticity R2 (Cutometer)

Change in skin elasticity at the immediate, 24 hour, and 2 week post application interval is shown in Table 6. Positive difference indicates an improvement in skin elasticity. Individualized skin elasticity results are shown in Table 17.

TABLE 6

| Parameter | Immediate | 24 Hour | Week 2 |
|---|---|---|---|
| Mean Percent Difference From | 0.78% | −1.75% | 3.10% |
| Baseline | (NS) | (NS) | (NS) |
| Percent of Subjects with Change | 50.00% | 40.00% | 50.00% |
| | (NS) | (NS) | (NS) |

### C. Appearance of Varicose Veins (Clinical Grading)

A statistically significant improvement in the appearance of varicose veins at the 2 week post application interval was observed as shown in Table 7. Negative difference indicates an improvement in the appearance of varicose veins. Individualized results are shown in Table 8.

TABLE 7

| | 1 Hour* | 24 Hour* | Week 2 |
|---|---|---|---|
| Parameter | (N = 22) | (N = 29) | (N = 30) |
| Mean Percent Difference From | −4.19% | 0.00% | −7.51% |
| Baseline | (NS) | (NS) | (p ≤ 0.001) |
| Percent of Subjects with Change | 6.67% | 0.00% | 23.33% |
| | (NS) | (NS) | (NS) |

### Example 8: Individual Data from the Clinical Study

### A. Individualized Skin Firmness R0 (Cutometer)

TABLE 8

| | R0 | | | | | | | |
|---|---|---|---|---|---|---|---|---|
| | Baseline | | Immediate | | 24 Hour | | Week 2 | |
| ID | Right | Left | Right | Left | Right | Left | Right | Left |
| 9918 | 0.343 | 0.221 | 0.320 | 0.211 | 0.208 | 0.233 | 0.271 | 0.259 |
| 10735 | 0.223 | 0.147 | 0.278 | 0.161 | 0.300 | 0.189 | 0.259 | 0.162 |
| 14742 | 0.292 | 0.363 | 0.371 | 0.224 | 0.306 | 0.252 | 0.487 | 0.447 |
| 15586 | 0.235 | 0.226 | 0.383 | 0.223 | 0.262 | 0.215 | 0.278 | 0.192 |
| 17557 | 0.211 | 0.340 | 0.276 | 0.435 | 0.315 | 0.438 | 0.323 | 0.417 |
| 18862 | 0.293 | 0.159 | 0.204 | 0.191 | 0.295 | 0.179 | 0.322 | 0.275 |
| 21767 | 0.443 | 0.385 | 0.442 | 0.346 | 0.404 | 0.401 | 0.475 | 0.426 |
| 21917 | 0.345 | 0.416 | 0.374 | 0.349 | 0.286 | 0.301 | 0.290 | 0.273 |
| 22674 | 0.351 | 0.420 | 0.375 | 0.465 | 0.309 | 0.381 | 0.464 | 0.491 |
| 22798 | 0.273 | 0.326 | 0.266 | 0.297 | 0.200 | 0.320 | 0.252 | 0.248 |
| 24464 | 0.558 | 0.492 | 0.540 | 0.516 | 0.503 | 0.467 | 0.554 | 0.493 |
| 24588 | 0.303 | 0.269 | 0.357 | 0.356 | 0.347 | 0.292 | 0.292 | 0.289 |
| 24618 | 0.305 | 0.349 | 0.358 | 0.385 | 0.336 | 0.358 | 0.338 | 0.350 |
| 30016 | 0.137 | 0.125 | 0.148 | 0.156 | 0.195 | 0.175 | 0.462 | 0.266 |
| 32682 | 0.260 | 0.472 | 0.320 | 0.375 | 0.270 | 0.322 | 0.345 | 0.306 |
| 32779 | 0.237 | 0.240 | 0.316 | 0.297 | 0.249 | 0.302 | 0.262 | 0.385 |
| 33468 | 0.175 | 0.159 | 0.236 | 0.229 | 0.230 | 0.237 | 0.305 | 0.243 |
| 33597 | 0.230 | 0.274 | 0.254 | 0.229 | 0.264 | 0.220 | 0.219 | 0.166 |
| 33902 | 0.082 | 0.121 | 0.124 | 0.219 | 0.117 | 0.207 | 0.139 | 0.190 |
| 35927 | 0.084 | 0.203 | 0.139 | 0.233 | 0.141 | 0.185 | 0.165 | 0.194 |
| 36093 | 0.314 | 0.417 | 0.320 | 0.407 | 0.349 | 0.387 | 0.304 | 0.331 |
| 36230 | 0.135 | 0.131 | 0.177 | 0.109 | 0.094 | 0.086 | 0.164 | 0.148 |
| 36724 | 0.238 | 0.195 | 0.255 | 0.197 | 0.241 | 0.192 | 0.170 | 0.145 |
| 36831 | 0.082 | 0.185 | 0.149 | 0.214 | 0.106 | 0.141 | 0.243 | 0.270 |
| 37951 | 0.228 | 0.229 | 0.274 | 0.237 | 0.205 | 0.290 | 0.221 | 0.235 |
| 37963 | 0.285 | 0.353 | 0.391 | 0.414 | 0.283 | 0.350 | 0.272 | 0.353 |
| 38566 | 0.150 | 0.180 | 0.142 | 0.190 | 0.167 | 0.143 | 0.158 | 0.134 |
| 38616 | 0.201 | 0.308 | 0.274 | 0.274 | 0.275 | 0.235 | 0.207 | 0.180 |
| 38655 | 0.170 | 0.236 | 0.216 | 0.209 | 0.145 | 0.184 | 0.133 | 0.135 |
| 38724 | 0.316 | 0.231 | 0.348 | 0.320 | 0.480 | 0.269 | 0.570 | 0.312 |

### B. Individualized Skin Elasticity R2 (Cutometer)

TABLE 9

| | R2 | | | | | | | |
|---|---|---|---|---|---|---|---|---|
| | Baseline | | Immediate | | 24 Hour | | Week 2 | |
| ID | Right | Left | Right | Left | Right | Left | Right | Left |
| 9918 | 0.7493 | 0.7557 | 0.7906 | 0.7441 | 0.6587 | 0.7425 | 0.6974 | 0.8069 |
| 10735 | 0.8117 | 0.7823 | 0.7914 | 0.6584 | 0.8133 | 0.6931 | 0.8494 | 0.7469 |
| 14742 | 0.7329 | 0.6143 | 0.8814 | 0.7277 | 0.6863 | 0.6706 | 0.8686 | 0.9687 |
| 15586 | 0.8340 | 0.4867 | 0.6815 | 0.7803 | 0.6756 | 0.5767 | 0.7230 | 0.6406 |
| 17557 | 0.7204 | 0.7882 | 0.6993 | 0.8299 | 0.8127 | 0.7945 | 0.8266 | 0.8777 |
| 18862 | 0.8259 | 0.5849 | 0.7402 | 0.6859 | 0.7864 | 0.5642 | 0.8075 | 0.6545 |
| 21767 | 0.8781 | 0.8519 | 0.7851 | 0.7428 | 0.8193 | 0.7805 | 0.8632 | 0.8779 |
| 21917 | 0.7942 | 0.7885 | 0.7139 | 0.7221 | 0.7448 | 0.7110 | 0.7759 | 0.7656 |
| 22674 | 0.5755 | 0.6881 | 0.7253 | 0.7785 | 0.6861 | 0.7270 | 0.8405 | 0.8024 |
| 22798 | 0.7326 | 0.8006 | 0.7632 | 0.7239 | 0.7000 | 0.7313 | 0.8016 | 0.8145 |
| 24464 | 0.8226 | 0.8760 | 0.7685 | 0.7500 | 0.7336 | 0.8801 | 0.8195 | 0.8337 |
| 24588 | 0.6832 | 0.6617 | 0.6387 | 0.6039 | 0.6397 | 0.7158 | 0.6952 | 0.6644 |
| 24618 | 0.8197 | 0.8424 | 0.7961 | 0.8286 | 0.7649 | 0.9190 | 0.8284 | 0.7571 |
| 30016 | 0.5620 | 0.5360 | 0.5676 | 0.7949 | 0.6564 | 0.6686 | 0.8896 | 0.8647 |
| 32682 | 0.8154 | 0.8559 | 0.7750 | 0.7840 | 0.7630 | 0.8727 | 0.8464 | 0.7941 |
| 32779 | 0.8059 | 0.5208 | 0.7911 | 0.7677 | 0.7871 | 0.6821 | 0.9771 | 0.8805 |
| 33468 | 0.4571 | 0.4528 | 0.5763 | 0.5939 | 0.5652 | 0.5485 | 0.7443 | 0.5761 |
| 33597 | 0.7478 | 0.7664 | 0.7559 | 0.6201 | 0.8258 | 0.8364 | 0.7352 | 0.6747 |
| 33902 | 0.7561 | 0.6033 | 0.5645 | 0.7352 | 0.6154 | 0.6232 | 0.3885 | 0.6053 |
| 35927 | 0.5357 | 0.6010 | 0.7482 | 0.6009 | 0.5390 | 0.5405 | 0.5273 | 0.5361 |
| 36093 | 0.8758 | 0.8777 | 0.8406 | 0.8305 | 0.8797 | 0.9018 | 0.8421 | 0.8520 |
| 36230 | 0.8963 | 0.8397 | 0.7345 | 0.6972 | 0.6277 | 0.8721 | 0.7378 | 0.7297 |
| 36724 | 0.7857 | 0.7590 | 0.8078 | 0.7614 | 0.7469 | 0.7917 | 0.7235 | 0.7103 |
| 36831 | 0.5610 | 0.5405 | 0.6779 | 0.8925 | 0.6604 | 0.7447 | 0.5432 | 0.6333 |
| 37951 | 0.6974 | 0.6812 | 0.6825 | 0.6329 | 0.4537 | 0.7310 | 0.5068 | 0.6426 |
| 37963 | 0.9649 | 0.7620 | 0.7852 | 0.8454 | 0.7880 | 0.8200 | 0.8640 | 0.8499 |
| 38566 | 0.7333 | 0.7722 | 0.7606 | 0.7842 | 0.6467 | 0.6993 | 0.7595 | 0.6716 |
| 38616 | 0.7015 | 0.8117 | 0.7372 | 0.7883 | 0.6571 | 0.7407 | 0.7778 | 0.8056 |
| 38655 | 0.9941 | 0.7712 | 0.6759 | 0.7225 | 0.6552 | 0.7446 | 0.6541 | 0.6519 |
| 38724 | 0.7373 | 0.6017 | 0.7644 | 0.7781 | 0.8438 | 0.5576 | 0.8228 | 0.8173 |

### C. Individualized Clinical Grading

TABLE 10

| | Appearance of Varicose Veins | | | |
|---|---|---|---|---|
| ID | Baseline | 1 Hour | 24 Hour | Week 2 |
| 9918 | 6.5 | 6.5 | 6.5 | 4.5 |
| 10735 | 6.5 | 7.0 | 7.0 | 6.5 |
| 14742 | 5.5 | 5.5 | 5.5 | 5.5 |
| 15586 | 1.5 | * | 1.5 | 1.5 |
| 17557 | 6.5 | 6.5 | 6.5 | 6.0 |
| 18862 | 7.0 | 7.0 | 7.0 | 6.0 |
| 21767 | 4.0 | 4.0 | 4.0 | 3.5 |
| 21917 | 7.5 | 5.0 | 7.5 | 5.0 |
| 22674 | 2.5 | 2.5 | 2.5 | 2.5 |
| 22798 | 4.5 | * | 4.5 | 4.5 |
| 24464 | 5.0 | 5.0 | 5.0 | 5.0 |
| 24588 | 4.0 | 4.0 | 4.0 | 3.0 |
| 24618 | 4.5 | 4.5 | 4.5 | 4.5 |
| 30016 | 3.0 | * | * | 3.0 |
| 32682 | 4.0 | 4.0 | 4.0 | 4.0 |
| 32779 | 8.0 | 8.0 | 8.0 | 7.5 |
| 33468 | 7.0 | 7.0 | 7.0 | 7.0 |
| 33597 | 2.0 | 2.0 | 2.0 | 2.0 |
| 33902 | 7.5 | 7.0 | 7.5 | 7.5 |
| 35927 | 6.5 | * | 6.5 | 6.5 |
| 36093 | 4.5 | * | 4.5 | 4.5 |
| 36230 | 4.0 | | 4.0 | 3.5 |
| 36724 | 6.5 | 5.0 | 6.5 | 6.5 |
| 36831 | 6.0 | * | 6.0 | 6.0 |
| 37951 | 7.5 | 7.5 | 7.5 | 7.5 |
| 37963 | 4.5 | 4.5 | 4.0 | 4.0 |
| 38566 | 1.5 | * | 1.5 | 0.0 |
| 38616 | 5.0 | 5.0 | 5.0 | 5.0 |
| 38655 | 1.5 | 1.0 | 1.5 | 1.5 |
| 38724 | 2.0 | 2.0 | 2.0 | 1.5 |
| Mean | 4.88 | 5.02 | 4.95 | 4.52 |
| SD | 2.02 | 1.95 | 2.04 | 2.02 |

*Clinical Grading not feasible due to erythema

### D. Post-Treatment Questionnaire (Immediate)

Results of the questionnaire completed by the subjects at the immediate post-application interval are shown in Tables 11 and 12.
Key: NR=No Response; N=Not Applicable.
Questions 1-4 Scale 1=No reduction; 5=Significant Reduction
Questions 6-7 Scale, 1-3=Mild, 4-7=Moderate, 8-10=Severe

TABLE 11

| | | | | | 5. After product | | |
|---|---|---|---|---|---|---|---|
| | | 2. The skin | | | application if you | | *7. Rate the |
| | 1. Was there a | tone in the | | | experienced any redness | | level of pain |
| | visible reduction | treated area | | | and/or a warming sensation | *6. Prior to any | or discomfort |
| | in the appearance | appears more | 3. Skin in the | 4. Treated Area | in the treated area, please | treatment, rate the | caused by your |
| | of varicose veins | even that it | treated area has | overall has a | state how long it | level of discomfort | varicose veins |
| | in the treated | was prior to | a firmer/tighter | more youthful | took for this reaction to | or pain caused by | on hour after |
| ID | area? | product use. | look and feels. | appearance. | subside. (minutes) | your varicose veins. | product application. |
| 9918 | 2 | 3 | 3 | 2 | NA | NR | NR |
| 10735 | 1 | 1 | 5 | 3 | 5 | 5 | 1 |
| 14742 | 1 | 1 | 1 | 1 | Still red after 1 hour | 1 | 1 |
| 15586 | 1 | 1 | 2 | 1 | NR | 1 | 6 |
| 17557 | 2 | 3 | 5 | 4 | 5 | NR | NR |
| 18862 | 2 | 4 | 3 | 2 | 3 | 1 | 1 |
| 21767 | 3 | 2 | 2 | 2 | 10 | 1 | 1 |
| 21917 | 1 | 1 | 1 | 1 | 1 | 7 | 7 |
| 22674 | 2 | 3 | 3 | 3 | 5 | 2 | 1 |
| 22798 | 3 | 3 | 3 | 2 | 40 | 1 | 1 |
| 24464 | 1 | 1 | 1 | 1 | NA | NR | NR |
| 24588 | 2 | 1 | 3 | 3 | NA | 5 | 5 |
| 24618 | 1 | 2 | 1 | 1 | 60 | 4 | 3 |
| 30016 | 1 | 1 | 1 | 1 | 10 | 9 | 8 |
| 32682 | 5 | 5 | 5 | 5 | NR | 5 | 5 |
| 32779 | 1 | 1 | 3 | 1 | 20 | 2 | 1 |
| 33468 | 3 | 2 | 3 | 3 | 60 | 4 | 4 |
| 33597 | 2 | 3 | 3 | 2 | 2 | 2 | 4 |
| 33902 | 1 | 1 | 1 | 1 | Still red | NR | NR |
| 35927 | 4 | 3 | 1 | 1 | 10 | NR | NR |
| 36093 | 1 | 1 | 2 | 1 | 60 | NR | NR |
| 36230 | 1 | 3 | 4 | 3 | 60 | 1 | 1 |
| 36724 | 2 | 2 | 3 | 2 | 2 | NR | NR |
| 36831 | 4 | 3 | 3 | 3 | 30 | 7 | 4 |
| 37951 | 1 | 2 | 5 | 2 | 2 | 5 | 4 |
| 37963 | 2 | 2 | 3 | 2 | 5 | 2 | 1 |
| 38566 | 3 | 3 | 3 | 3 | 10 | 2 | 2 |
| 38616 | 1 | 2 | 3 | 3 | 2 | NR | NR |
| 38655 | 3 | 3 | 3 | 3 | 45 | 1 | 1 |
| 38724 | 5 | 5 | 5 | 5 | 0 | 4 | 4 |

TABLE 12

| | | | 10. Any additional |
|---|---|---|---|
| | | | comments about your |
| | 8. Please tell us what you | 9. Please tell us what you | experience using the |
| ID | like best about the product. | like least about the product. | product: |
| 9918 | Easy to apply | Made the area red | NA |
| 10735 | It goes on smooth | NR | NR |
| 14742 | NA | I didn't notice any visible | NA |
| | | difference | |
| 15586 | Roll on application . . . | The redness + slight | NA |
| | works well when applying | burning | |
| | to leg arcas | | |
| 17557 | I like that it's a roll on- | The smell/odor | NA |
| | less mess | | |
| 18862 | NA | NA | NA |
| 21767 | NA | It made my skin feel warm + | NA |
| | | turned red in the area | |
| | | where I applied the product | |
| 21917 | NA | NA | NA |
| 22674 | Decreases the appearance | The smell of the product | NA |
| | of varicose veins | | |
| 22798 | NA | NA | NA |
| 24464 | To soon to tell | The redness | No |
| 24588 | No pain or stinging | Not sure how well it is | Need more time using |
| | | working | product to make an |
| | | | assessment |
| 24618 | NA | NA | NA |
| 30016 | I don't like the product at | NA | I feel itchy and my skin |
| | all | | became red and swollen |
| 32682 | NA | NA | NA |
| 32779 | NA | NA | NA |
| 33468 | Easy application | Redness occurs after use. | Felt minor tingling & |
| | | | tightening of skin area. |
| 33597 | NA To soon | NA too soon | NA |
| 33902 | First time using it so can't | Makes my skin red | nothing except the red skin |
| | really say except that it is | | |
| | easy to use | | |
| 35927 | Don't know yet | The fragrance | NO |
| 36093 | NA | NA | NA |
| 36230 | They started to look better | Nothing yet | I'm hopeful in the end my |
| | | | legs will look better and I |
| | | | can wear shorts again |
| 36724 | NA | Redness | NA |
| 36831 | It seems to work | The red color | Hope it really works. |
| 37951 | May help my varicose | Not sure of yet | Looking forward to the |
| | veins | | progress |
| 37963 | Cool on skin | NA | NA |
| 38566 | To early to tell | The redness and burning | Nothing to report |
| 38616 | NA | NA | NA |
| 38655 | NA | Burning sensation | NA |
| 38724 | NA | NA | NA |

### E. Post-Treatment Questionnaire (8 Hour)

Results of the questionnaire completed by the subjects at the 8 hour post-application interval are shown in Tables 13 and 14.
Key: NR:=No Response, NA=Not Applicable.
Questions 1-4: Scale 1=No reduction; 5==Significant Reduction
Questions 6-7: Scale, 1-3=Mild, 4-7=Moderate, 8-10=Severe

TABLE 13

| | | | | | 5. After product | | |
|---|---|---|---|---|---|---|---|
| | | 2. The skin | | | application if you | | *7. Rate the |
| | 1. Was there a | tone in the | | | experienced any redness | | level of pain |
| | visible reduction | treated area | | | and/or a warming sensation | *6. Prior to any | or discomfort |
| | in the appearance | appears more | 3. Skin in the | 4. Treated Area | in the treated area, please | treatment, rate the | caused by your |
| | of varicose veins | even that it | treated area has | overall has a | state how long it | level of discomfort | varicose veins |
| | in the treated | was prior to | a firmer/tighter | more youthful | took for this reaction to | or pain caused by | on hour after |
| ID | area? | product use. | look and feels. | appearance. | subside. (minutes) | your varicose veins. | product application. |
| 9918 | 2 | 2 | 2 | 1 | All night long | NR | NR |
| 10735 | 5 | 5 | 5 | 5 | 5-10 | 5 | 1 |
| 14742 | 2 | 1 | 2 | 2 | 10 | 6 | 5 |
| 15586 | 2 | 1 | 4 | 1 | 90 | 1 | 1 |
| 17557 | 3 | 4 | 4 | 4 | 15 | NR | NR |
| 18862 | 3 | 3 | 3 | 2 | 2 | 1 | 1 |
| 21767 | 2 | 1 | 1 | 1 | 20 | 1 | 1 |
| 21917 | 1 | 1 | 2 | 1 | 180 | 7 | 6 |
| 22674 | 3 | 3 | 3 | 3 | 5 | 3 | 1 |
| 22798 | 4 | 4 | 4 | 3 | 40 | 2 | 1 |
| 24464 | 2 | 1 | 2 | 2 | 40 | 1 | 1 |
| 24588 | 4 | 3 | 3 | 3 | 15 | 6 | 6 |
| 24618 | 1 | 1 | 1 | 1 | 60 | 7 | 5 |
| 30016 | 1 | 4 | 2 | 2 | 10 | 6 | 6 |
| 32682 | 4 | 4 | 4 | 4 | NA | 5 | 5 |
| 32779 | 1 | 1 | 1 | 1 | 20 | 3 | 2 |
| 33468 | 4 | 4 | 3 | 3 | 60 | 4 | 4 |
| 33597 | 1 | 3 | 3 | 2 | 4 | 2 | 1 |
| 33902 | 1 | 1 | 2 | 1 | 120 | NR | NR |
| 35927 | 1 | 1 | 1 | 1 | 10 | 1 | 1 |
| 36093 | 1 | 1 | 2 | 1 | 150 | NR | NR |
| 36230 | 4 | 4 | 4 | 4 | NR | 8 | 3 |
| 36724 | 2 | 2 | 2 | 1 | 5 | NR | NR |
| 36831 | 3 | 4 | 4 | 4 | 30 | 8 | 4 |
| 37951 | 4 | 3 | 2 | 2 | 15 | 8 | 5 |
| 37963 | 2 | 2 | 3 | 3 | 5 | 1 | 1 |
| 38566 | 4 | 4 | 4 | 4 | 13 | 1 | 1 |
| 38616 | 2 | 2 | 2 | 2 | NA | 1 | 1 |
| 38655 | 5 | 5 | 5 | 5 | 5 | 1 | 1 |
| 38724 | 3 | 3 | 4 | 5 | 15 | 5 | 5 |

TABLE 14

| | | | 10. Any additional |
|---|---|---|---|
| | 8. Please tell us what you | 9. Please tell us what you | comments about your |
| | like best about the | like least about the | experience using the |
| ID | product. | product. | product: |
| 9918 | Easy to apply | Burning + redness | NA |
| 10735 | It is easy to apply and | Nothing I don't like least | I do believe this is the |
| | spreads evenly | about the product | treatment for varicose |
| | | | veins |
| 14742 | NA | NA | NA |
| 15586 | Application | The redness & warming | Second application not |
| | | sensation . . . felt like a | like the 1st application |
| | | sunburn a little | |
| 17557 | The youthful appearance | The smell | NA |
| | it makes on the skin | | |
| 18862 | NA | NA | NA |
| 21767 | It is a roll-on | The warm sensation after | No |
| | | I apply the product on my | |
| | | varicose veins | |
| 21917 | NA | If this is a laser treatment | NA |
| | | and feel discomfort | |
| | | afterwards, I totally | |
| | | understand but it is just a | |
| | | lotion, some might think | |
| | | if it's safe such as PFAS | |
| 22674 | Fading of the varicose | The smell | NA |
| | veins | | |
| 22798 | It diminished the look of | The redness after | NA |
| | my varicose veins | application | |
| 24464 | Roll on. | Turned skin red | NR |
| 24588 | Seems to lessen the | NA | NA |
| | buldging of my veins | | |
| 24618 | NA | NA | NR |
| 30016 | NA | NA | NA |
| 32682 | NA | NA | NA |
| 32779 | NA | NA | NA |
| 33468 | Ease of application | redness after use | So far no other |
| | | | symptoms/irritation |
| 33597 | NA | NA | NA |
| 33902 | Easy to use | Tums your skin red has a | Easy to use which is some |
| | | funny smell | things I like in a product |
| | | | and goes on easily |
| 35927 | Nothing | The fragrance | You need to be using the |
| | | | product for about a month |
| | | | or so to notice a |
| | | | difference |
| 36093 | NA | NA | NA |
| 36230 | It looks better and smaller | The pain related | NA |
| 36724 | Goes on easily; doesn't | Redness | NA |
| | smell bad-would love to | | |
| | see outcome if used | | |
| | longer | | |
| 36831 | NA | The redness but went | NA |
| | | away about 2 hrs or so | |
| | | after applying | |
| 37951 | Don't see any different | Day 1 not sure has to use | Discomfort and redness in |
| | | the product to give a | my skin. And irritation |
| | | better answer | |
| 37963 | Feels cool | NA | Feel good |
| 38566 | I believe it lights veins | The bum | NA |
| 38616 | NA | NA | NA |
| 38655 | That it made varicose | NA | NA |
| | veins disappear | | |
| 38724 | None | NA | NA |

### Post-Treatment Questionnaire (24 Hour)

Results of the questionnaire completed by the subjects at the 24 hour post-application interval are shown in Tables 15 and 16.
Key: NR=No Response; NA=Not Applicable.
Questions 1-4: Scale 1=No reduction; 5=Significant Reduction
Questions 6-7: Scale; 1-3=Mild, 4-7=Moderate, 8-10=Severe

TABLE 15

| | | | | | 5. After product | | |
|---|---|---|---|---|---|---|---|
| | | 2. The skin | | | application if you | | *7. Rate the |
| | 1. Was there a | tone in the | | | experienced any redness | | level of pain |
| | visible reduction | treated area | | | and/or a warming sensation | *6. Prior to any | or discomfort |
| | in the appearance | appears more | 3. Skin in the | 4. Treated Area | in the treated area, please | treatment, rate the | caused by your |
| | of varicose veins | even that it | treated area has | overall has a | state how long it | level of discomfort | varicose veins |
| | in the treated | was prior to | a firmer/tighter | more youthful | took for this reaction to | or pain caused by | on hour after |
| ID | area? | product use. | look and feels. | appearance. | subside. (minutes) | your varicose veins. | product application. |
| 9918 | 2 | 2 | 2 | 1 | 180 | NR | NR |
| 10735 | 5 | 5 | 5 | 5 | 5-10 | 5 | 1 |
| 14742 | 2 | 1 | 2 | 2 | NA | 6 | 5 |
| 15586 | 2 | 1 | 1 | 1 | 90 | 1 | 1 |
| 17557 | 3 | 4 | 4 | 4 | NA | NR | NR |
| 18862 | 3 | 2 | 3 | 2 | 2 | 1 | 1 |
| 21767 | 2 | 1 | 1 | 1 | 15 | 1 | 1 |
| 21917 | 1 | 1 | 1 | 1 | 180 | 1 | 1 |
| 22674 | 3 | 3 | 3 | 3 | 5 | 2 | 1 |
| 22798 | 4 | 4 | 4 | 4 | 35 | 1 | 1 |
| 24464 | 2 | 1 | 1 | 1 | 40 | 1 | 1 |
| 24588 | 4 | 3 | 3 | 3 | 15 | 6 | 6 |
| 24618 | 1 | 1 | 1 | 1 | 960 | 6 | 4 |
| 30016 | 1 | 2 | 2 | 2 | 10 | 6 | 6 |
| 32682 | 4 | 4 | 5 | 5 | NA | 5 | 5 |
| 32779 | 1 | 1 | 1 | 1 | NA | 2 | 2 |
| 33468 | 4 | 4 | 4 | 4 | Overnight | 4 | 4 |
| 33597 | 1 | 2 | 1 | 1 | 4 | 1 | 4 |
| 33902 | 1 | 1 | 2 | 1 | 35 | NR | NR |
| 35927 | 1 | 1 | 1 | 1 | 10 | 1 | 1 |
| 36093 | 1 | 1 | 1 | 1 | NA | NR | NR |
| 36230 | 4 | 4 | 4 | 4 | NR | NR | NR |
| 36724 | 2 | 3 | 2 | 1 | 5 | NR | NR |
| 36831 | 5 | 4 | 4 | 3 | NA | 8 | NR |
| 37951 | 5 | 2 | 2 | 2 | 15 | 7 | 7 |
| 37963 | 3 | 3 | 3 | 3 | 5 | 1 | 1 |
| 38566 | 4 | 4 | 4 | 4 | 15 | 1 | 1 |
| 38616 | 2 | 2 | 2 | 2 | NA | 2 | 2 |
| 38655 | 3 | 3 | 3 | 3 | NA | NR | NR |
| 38724 | 5 | 5 | 5 | 5 | NA | 10 | 10 |

TABLE 16

| | | | 10. Any additional |
|---|---|---|---|
| | | 9. Please tell us what you | comments about your |
| | 8. Please tell us what you | like least about the | experience using the |
| ID | like best about the product. | product. | product: |
| 9918 | Easy to apply | Redness burning | NA |
| 10735 | The smell and the | Nothing I don't like | It really seems to work |
| | application goes on smooth | | |
| 14742 | NA | NA | NA |
| 15586 | The way in which you are | The redness after applying | NR |
| | able to apply the product | the product seems to be | |
| | | less red than first | |
| | | application | |
| 17557 | I like the youthfulness and | The smell/odor. Wish it | Maybe the scent |
| | firming of my legs | was a more pleasant | |
| | | fragrance | |
| 18862 | NA | NA | NA |
| 21767 | NA | The warming sensation | NA |
| | | after I apply the product | |
| 21917 | NA | NA | NA |
| 22674 | The fading of the varicose | The smell and the redness | None |
| | veins | it produces on my skin | |
| 22798 | NA | NA | NA |
| 24464 | Easy to apply | Redness after applying | NR |
| 24588 | Easy to apply | NA | NA |
| 24618 | NA | NA | NR |
| 30016 | NA | NA | NA |
| 32682 | NA | NA | NA |
| 32779 | NA | NA | NA |
| 33468 | Easy to apply | Redness & tingling | None |
| 33597 | NA too soon | NA too soon | NA |
| 33902 | easy to use | smells funny and turns skin | Just that it is easy to use |
| | | red. | |
| 35927 | Nothing | The fragrance is awful-no | Nope |
| | | one will buy it I sure | |
| | | won't!! | |
| 36093 | NA | NA | NA |
| 36230 | The look that they might | the stinging after you apply | NR |
| | be better | to vein | |
| 36724 | Very easy to use | NA | NA |
| 36831 | It seems to be working | the redness likely it goes | NA |
| | | away after a couple of hrs. | |
| 37951 | Not working yet | Redness in varicose veins | Pain |
| | | area | |
| 37963 | Feel good | NA | NA |
| 38566 | They lightening a little | The burning | NA |
| 38616 | NA | NA | NA |
| 38655 | Seems to help lighten the | NA | NA |
| | varicose veins | | |
| 38724 | I like it | NA | NA |

### G. Post-Treatment Questionnaire (Week 2)

Results of the questionnaire completed by the subjects at the 2 week post-application interval are shown in Tables 17 and 18.
Key: NR=No Response; NA=Not Applicable
Questions 1-4: Scale 1=No reduction; 5=Significant Reduction
Questions 6-7: Scale; 1-3=Mild, 4-7=Moderate, 8-10=Severe

TABLE 17

| | | | | | 5. After product | | |
|---|---|---|---|---|---|---|---|
| | | 2. The skin | | | application if you | | *7. Rate the |
| | 1. Was there a | tone in the | | | experienced any redness | | level of pain |
| | visible reduction | treated area | | | and/or a warming sensation | *6. Prior to any | or discomfort |
| | in the appearance | appears more | 3. Skin in the | 4. Treated Area | in the treated area, please | treatment, rate the | caused by your |
| | of varicose veins | even that it | treated area has | overall has a | state how long it | level of discomfort | varicose veins |
| | in the treated | was prior to | a firmer/tighter | more youthful | took for this reaction to | or pain caused by | on hour after |
| ID | area? | product use. | look and feels. | appearance. | subside. (minutes) | your varicose veins. | product application. |
| 9918 | 3 | 3 | 3 | 3 | 60 | NR | NR |
| 10735 | 5 | 5 | 5 | 5 | 3 | 1 | 1 |
| 14742 | 2 | 2 | 2 | 2 | 5 | 5 | 4 |
| 15586 | 2 | 2 | 3 | 2 | 30 | 1 | 1 |
| 17557 | 2 | 4 | 4 | 4 | 1 | 5 | 4 |
| 18862 | 4 | 3 | 3 | 3 | 2 | 5 | 3 |
| 21767 | 3 | 2 | 2 | 2 | 2 | 1 | 2 |
| 21917 | 1 | 1 | 1 | 1 | 300 | 8 | 5 |
| 22674 | 4 | 4 | 4 | 4 | 10 | 2 | 1 |
| 22798 | 4 | 5 | 4 | 4 | 35 | 2 | 2 |
| 24464 | 3 | 2 | 3 | 3 | 50 | 1 | 1 |
| 24588 | 4 | 3 | 4 | 3 | 5 | 7 | 7 |
| 24618 | 1 | 1 | 1 | 1 | 25 | 8 | 6 |
| 30016 | 1 | 2 | 3 | 2 | 15 | 9 | 1 |
| 32682 | 5 | 5 | 5 | 4 | 10 | 5 | 5 |
| 32779 | 4 | 4 | 4 | 4 | 15 | 3 | 3 |
| 33468 | 4 | 4 | 4 | 4 | 20 | 4 | 3 |
| 33597 | 2 | 2 | 3 | 1 | 10 | 1 | 2 |
| 33902 | 2 | 1 | 2 | 2 | 20 | NR | NR |
| 35927 | 2 | 2 | 3 | 1 | 10 | 1 | 2 |
| 36093 | 2 | 2 | 1 | 1 | 5 | NR | NR |
| 36230 | 4 | 4 | 4 | 4 | 15 | 1 | 4 |
| 36724 | 3 | 3 | 3 | 3 | 5 | NR | NR |
| 36831 | 5 | 5 | 4 | 4 | About 20 | 7 | 3 |
| 37951 | 2 | 3 | 2 | 4 | 5 | 4 | 4 |
| 37963 | 3 | 3 | 3 | 3 | 5 | 7 | 3 |
| 38566 | 3 | 3 | 2 | 2 | 5 | 1 | 1 |
| 38616 | 2 | 2 | 2 | 2 | 5 | 2 | 2 |
| 38655 | 2 | 2 | 3 | 3 | 20 | 4 | 5 |
| 38724 | 5 | 5 | 5 | 5 | 15 | 5 | 4 |

TABLE 18

| | | | 10. Any additional |
|---|---|---|---|
| | | 9. Please tell us what you | comments about your |
| | 8. Please tell us what you | like least about the | experience using the |
| ID | like best about the product. | product. | product: |
| 9918 | Easy to apply | Redness + warming | N/A |
| 10735 | It really works | Nothing I didn't like | I cant wait until it comes |
| | | | to the stores I will surely |
| | | | purchase |
| 14742 | N/A | N/A | N/A |
| 15586 | I like the application | The redness . . . I didn't like | N/A |
| | process. Roll-on. Didn't | that it left the redness after | |
| | have to touch the product | immediate application. | |
| | | Didn't like the smell too | |
| | | much | |
| 17557 | It makes my legs look | The smell or odor | N/A |
| | firmer and younger more | | |
| | youthful appearance | | |
| 18862 | Some varicose veins are | Easy to use | I do not like smell |
| | reduced | | |
| 21767 | N/A | N/A | N/A |
| 21917 | N/A | N/A | I didn't feel/see the |
| | | | difference but burning, |
| | | | itching on my skin. Big |
| | | | Red spots like bruises are |
| | | | always on my skin. |
| 22674 | The product reduced the | The smell and the feel | Good product. |
| | appearance of the varicose | after putting the product | |
| | veins | on. It rapidly works. | |
| 22798 | I like the roll on | Redness on my legs. | I developed a rash on my |
| | applicator. | | upper leg + not on the |
| | | | varicose vein (part) |
| 24464 | Easy to apply. | Having to apply gain in 20 | No |
| | | minutes | |
| 24588 | Easy to use, no reaction | N/A | N/A |
| | after regular use. | | |
| 24618 | Easy to apply | Saw very little progress or | N/A |
| | | changes | |
| 30016 | N/A | N/A | Feels uncomfortable right |
| | | | after treat |
| 32682 | NA | NA | NA |
| 32779 | N/A | N/A | N/A |
| 33468 | Ease of application, visible | Had strong redness & | N/A |
| | reduction of vein swelling. | discomfort for 1^{st} day or so | |
| | | of using product. | |
| 33597 | N/A | N/A | N/A |
| 33902 | Easy to use | I really don't like the 20 | N/A |
| | | min wait time and the | |
| | | smell | |
| 35927 | Nothing | The awful fragrance | N/A |
| 36093 | NR | NR | NR |
| 36230 | Thee look | The feel after applying | N/A |
| 36724 | Very easy to apply, dried | Smell | Would love to be able to |
| | quickly | | use longer to really show a |
| | | | reduction |
| 36831 | It work I show my friends | Not been able to have a | I like to know the name of |
| | and they agreed it looks | pedicure & or massage. | it when it goes out in the |
| | better | That I had to do it and like | market |
| | | any medicine it won't | |
| | | work if you do not applie | |
| | | it | |
| 37951 | Have not seen any change. | When I apply the product | Redness, burning, |
| | From the product to better | in about 5 min I have | warming, irritation. |
| | veins on my | burning sensation. Can't | |
| | legs . . . problem. | lay down. On any thing | |
| | | have to stand up until until | |
| | | the burning goes away | |
| 37963 | It was cool and felt good | Turns red after applying | N/A |
| | | slight discomfort | |
| 38566 | It does reduce visiualabity | It smells like burnt flesh | Reduced smell and it |
| | | | would not be so bad |
| 38616 | N/A | N/A | N/A |
| 38655 | Seems to make the | that it burns & make my | N/A |
| | varicose veins slightly. | skin red | |
| 38724 | It's perfect | I like it. | It's ok. |

Although topical formulations have been disclosed in the context of certain embodiments and examples, it will be understood by those skilled in the art that the composition and arrangement of the ingredients extends beyond the specifically disclosed embodiments to other alternative embodiments and/or uses of the embodiments and certain modifications and equivalents thereof. It should be understood that various features and aspects of the disclosed embodiments can be combined with or substituted for one another in order to form varying embodiments of the formulations (e.g., skin healing formulations) provided herein, Additional combinations and features are also contemplated by the inventors. Any ingredients of the disclosed formulations may be mixed and matched to provide a number of formulations with desired properties. Thus, the scope of the inventions herein-disclosed should not be limited by the particular disclosed embodiments described above or in the claims.

1-31. (canceled)

32. A skin healing formulation for ameliorating or treating a vascular network of a subject comprising:
a) at least one bioflavonoid;
b) at least one saponin; and
c) at least one skin soothing agent.

33. The skin healing formulation of claim 32, wherein the at least one bioflavonoid is troxerutin.

34. The skin healing formulation of claim 32, wherein the at least one saponin is selected from the group consisting of allantoin, ammonium glycyrrhizate, escin, ruscus aculeatus root, and any combination of the foregoing.

35. The skin healing formulation of claim 32, wherein the at least one skin soothing agent comprises acacia senegal gum, glycoproteins, polysaccharides, or any combination of the foregoing.

36. The skin healing formulation of claim 32, wherein the skin healing formulation further comprises at least one active botanical compound.

37. The skin healing formulation of claim 36, wherein the at least one active botanical compound is selected from a group consisting of Calendula officinalis, Centella sciatica, and any combination of the foregoing.

38. The skin healing formulation of claim 32, wherein the skin healing formulation further comprises:
at least one amino acid selected from the group consisting of arginine, aspartic acid, pyroglutamic acid, glycine, alanine, serine, valine, proline, threonine, isoleucine, histidine, phenylalanine, and any combination of the foregoing;
at least one antioxidant selected from the group consisting of tetrahexyldecyl ascorbate, tocopherol and any combination of the foregoing;
at least one emollient selected from the group consisting of butylene glycol, caprylic triglyceride, capric triglyceride, and any combination of the foregoing;
at least one emulsifier wherein the at least one emulsifier is polysorbate 80;
at least one fragrance source wherein the at least one fragrance source is Vibrance Type FW;
at least one humectant selected from the group consisting of glycerine, propylene glycol, propanediol, sodium lactate, sodium pyroglutamic acid, and any combination of the foregoing;
at least one moisturizer selected from the group consisting of panthenol, hydrolyzed yeast protein, cholecalciferol, and any combination of the foregoing;
at least one rubifacient wherein the at least one rubifacient is methyl nicotinate; and
at least one antimicrobial selected from the group consisting of phenyl propanol, caprylyl glycol and any combination of the foregoing.

39. The skin healing formulation of claim 32, wherein the at least one bioflavonoid is troxerutin, the at least one saponin are escin and ruscus aculeatus root, and the at least one skin soothing agent is acacia senegal gum, and based on a total weight of troxerutin, escin, ruscus aculeatus root, and acacia senegal gum,
troxerutin is present at a weight percent from about 10% to about 98 wt %,
escin is present at a weight percent from about 0.5% to about 40 wt %,
ruscus aculeatus root is present at a weight percent from about 0.3% to about 20 wt %, and
acacia senegal gum is present at a weight percent from about 2% to about 40 wt %,
wherein a sum of components troxerutin, escin, ruscus aculeatus root, and acacia senegal gum adds up to 100 wt %.

40. The skin healing formulation of claim 32, wherein based on a total weight of the skin healing formulation,
the at least one bioflavonoid comprises troxerutin at a weight percent from about 0.01% to about 30%;
the at least one saponin comprises escin a weight percent from about 0.005% to about 5%, and ruscus aculeatus root at a weight percent from about 0.001% to about 3%; and
the at least one skin soothing agent comprises acacia senegal gum at a weight percent from about 0.02% to about 4%.

41. The skin healing formulation of claim 40, wherein based on a total weight of the skin healing formulation,
the at least one bioflavonoid comprises troxerutin at a weight percent from about 0.95% to about 1.05% and further comprises Ginkgo biloba at a weight percent from about 0.19% to about 0.21%;
the at least one saponin comprises escin at a weight percent from about 0.14% to about 0.16%, ruscus aculeatus root at a weight percent from about 0.074% to about 0.076%, and further comprises allantoin at a weight percent from about 0.38% to about 0.42% and ammonium glycyrrhizate at a weight percent from about 0.048% to about 0.052%;
the at least one skin soothing agent comprises acacia senegal gum at a weight percent from about 0.24% to about 0.26%, and further comprises xanthan gum at a weight percent from about 0.20% to about 0.22%;
wherein the skin healing formulation further comprises:
at least one active botanical compound comprising Calendula officinalis at a weight percent from about 0.017% to about 0.018% and centella asiatica at a weight percent from about 0.032% to about 0.033%;
arginine at a weight percent from about 0.038% to about 0.042%;
aspartic acid at a weight percent from about 0.025% to about 0.027%;
pyroglutamic acid at a weight percent from about 0.020% to about 0.022%;
glycine at a weight percent from about 0.0061% to about 0.0067%;
alanine at a weight percent from about 0.0058% to about 0.0062%;
serine at a weight percent from about 0.0038% to about 0.0042%;
valine at a weight percent from about 0.0031% to about 0.0033%;
proline at a weight percent from about 0.0019% to about 0.0021%;
threonine at a weight percent from about 0.0019% to about 0.0021%;
isoleucine at a weight percent from about 0.0019% to about 0.0021%;
histidine at a weight percent from about 0.00076% to about 0.00084%;
phenylalanine at a weight percent from about 0.00076% to about 0.00084%;
tetrahexyldecyl ascorbate at a weight percent from about 0.010% to about 0.012%;
tocopherol at a weight percent from about 0.001% to about 0.0012%;
butylene glycol at a weight percent from about 3.3% to about 3.7%;
caprylic triglyceride at a weight percent from about 2.4% to about 2.6%;
capric triglyceride at a weight percent from about 2.4% to about 2.6%;
polysorbate 80 at a weight percent from about 0.95% to about 1.05%;
Vibrance Type FW at a weight percent from about 0.16% to about 0.18%;
glycerin at a weight percent from about 6.1% to about 6.7%;
propylene glycol at a weight percent from about 2.4% to about 2.6%;
propanediol at a weight percent from about 0.26% to about 0.28%;
sodium lactate at a weight percent from about 0.057% to about 0.063%;
sodium pyroglutamic acid at a weight percent from about 0.071% to about 0.079%;
panthenol at a weight percent from about 0.48% to about 0.52%;
hydrolyzed yeast protein at a weight percent from about 0.019% to about 0.021%;
cholecalciferol at a weight percent from about 0.0000056% to about 0.000006%;
methyl nicotinate at a weight percent from about 1.05% to about 1.15%;
phenyl propanol at a weight percent from about 0.5% to about 0.6%;
caprylyl glycol at a weight percent from about 0.26% to about 0.3%; and
water at a weight percent from about 71.8% to about 86.8%.

42. A method of ameliorating or treating a vascular network of a subject in need thereof comprising administering the skin healing formulation of claim 1 to a subject in need thereof.

43. The method of claim 42, the method further comprising improving an appearance of varicose veins and/or reducing prominence of varicose veins of the subject.

44. The method of claim 42, the method further comprising alleviating or reducing pain of varicose veins of the subject.

45. The method of claim 42, wherein the at least one bioflavonoid is troxerutin.

46. The method of claim 42, wherein the at least one saponin is selected from the group consisting of allantoin, ammonium glycyrrhizate, escin, ruscus aculeatus root, and any combination of the foregoing.

47. The method of claim 42, wherein the at least one skin soothing agent is acacia senegal gum.

48. The method of claim 42, wherein the skin healing formulation further comprises at least one active botanical compound.

49. The method of claim 42, wherein the skin healing formulation is administered topically.

50. The method of claim 42, wherein a delay time after the skin healing formulation is administered such that an improved appearance begins is equal to or less than about 10 seconds to about 10 minutes.

51. The method of claim 42, wherein a time span after the skin healing formulation is administered such that an improved appearance ends is equal to or more than about 30 minutes to about 8 hours.
